SEQUENCE LISTING (2 sequences)

SEQ ID NO: 1 - DNA, 19 bp, Artificial Sequence
  npt2a primer
ccgccacacccagccggcc

SEQ ID NO: 2 - DNA, 18 bp, Artificial Sequence
  npt2s primer
ccgacctgtccggtgccc

(19) United States
(12) Patent Application Publication
(10) Pub. No.: US 2013/0102079 A1
(43) Pub. Date: Apr. 25, 2013

(54) POLYAMINE-CONTAINING POLYMERS AND METHODS OF SYNTHESIS AND USE

(71) Applicants: Robert W. Jost, St. Albert (CA); Hasan Uludag, Edmonton (CA); Mehmet Yaman Boluk, Edmonton (CA)

(72) Inventors: Robert W. Jost, St. Albert (CA); Hasan Uludag, Edmonton (CA); Mehmet Yaman Boluk, Edmonton (CA)

(73) Assignee: Alberta Innovates - Technology Futures, Edmonton (CA)

(21) Appl. No.: 13/723,933

(22) Filed: Dec. 21, 2012

Related U.S. Application Data
(62) Division of application No. 13/078,347, filed on Apr. 1, 2011.
(60) Provisional application No. 61/320,355, filed on Apr. 2, 2010.

(51) Int. Cl. C08B 37/02; C12N 15/64; C08F 16/06; C08B 15/06; C08F 20/14

(52) U.S. Cl. CPC C08B 37/0021; C08B 15/06; C08F 20/14; C08F 16/06; C12N 15/64
USPC 435/455; 435/468

(57) ABSTRACT

The present invention relates to polyamine-containing polymers and methods of their synthesis and use. The polymer may be hydroxyethylcellulose, dextran, poly(vinyl alcohol) or poly(methyl acrylate).

### CROSS REFERENCE TO RELATED APPLICATIONS

This application is a divisional application of U.S. application Ser. No. 13/078,347 filed Apr. 1, 2011, which claims priority upon U.S. provisional application Ser. No. 61/320,355, filed Apr. 2, 2010. These applications are hereby incorporated by reference in their entireties.

### TECHNICAL FIELD

The present invention relates to compounds comprising carbon polymers and one or more polyamine groups.

### BACKGROUND

Nucleic acids encoding biologically active polypeptides or nucleic acids may be transferred to a cell by any of several methods, including viral vectors and chemical transfection. The choice of technique is a balance between the need to incorporate the nucleic acid efficiently, minimizing impact on the short term, and preferably the long term, survival of the cell, and without compromising the genetic makeup of the cell.

Aminated, cationic polymers that interact with the nucleic acid and are then taken up by the cell may be advantageous, at least, by avoiding some of the immunological and mutagenic concerns that accompany some viral transformation systems. DEAE-dextran is an example of an aminated polymer that is relatively non-toxic, however, the efficiency may be low. Polyethyleneimine (PEI) (Boussif, et al., 1995. Proc. Natl. Acad. Sci. 1995, 95, 7297-7301) has a high cationic charge density for DNA condensation, and also exhibits membrane-perturbing activity necessary for escape of internalized DNA from endosomal compartment. Branched, high molecular weight PEI (˜25 kDa) is an effective polymeric carrier. Transfection with PEI-nucleic acid complexes is significantly more efficient than observed with DEAE-dextran, however PEI demonstrates a dose-dependent cytotoxicity. Generally, both the transfection efficiency and cytotoxicity increase with the molecular weight of the PEI (Fischer D et al., 1999. Pharm Res., 16:1273-1279).

Other aminated polymers have been proposed seeking a balance between efficiency and cytotoxicity and include, for example, chitosan-EDTA (Loretz et al., 2006 AAPS Journal 8(4): E756-764), chitosan combined with PEI (Zhao et al., 2009 Biol. Pharm Bull 32(4): 706-710; Jiang, et al., 2007 J. Control. Release 117:273-280), and derivatized dentrimers (Mintzer et al., 2009. New J. Chem 33:1918-1925). Huang et al, (2006, Chem. Commun. 22:2382-2384) discloses low molecular weight PEI crosslinked with cyclodextrins, and also a method by which such polymers may be prepared. Wittmar et al., (2005, Bioconjug Chem 16(6):1390-8) describes transfection studies of an amine-modified poly(vinyl alcohol) for gene delivery. Other cationic polymers are described generally by Schmidt-Wolf (2003, Trend. Mol. Med. 2003, 9, 67).

Production of aminocellulose from methylcellulose is described in U.S. Pat. No. 2,136,299. This method employs a p-toluenesulfonyl chloride to activate the primary hydroxyl group. Reactions involving aromatic diamines or triamines with tosylcellulose (cellulose activated with p-toluenesulfonyl chloride, as per U.S. Pat. No. 2,136,299, or analogous reactions) are described in U.S. Pat. No. 6,358,754, and reductive amination of a hydroxyalkyl cellulose is described in U.S. Pat. No. 4,124,758. A variety of diamines may be attached to cellulose, to provide compositions of various properties; however, as a primary hydroxy is the site of reaction, the degree of substitution may be limited to 1. These compositions may be film-forming, solid or semi-solid according to the specifics of the amine and the intended use (Tiller et al., 1999. Macromol. Chem. Phys 200, 1-9; Berlin et al., 2000. Macromol Chem Phys 201, 2070-2082; Tiller et al, 2000. Appl. Polym Sci 75: 904-915; Becher et al., 2004. Cellulose 11:119-126).

US 2008/0177021 discloses a method of making solid, composite substrates formed from aminocellulose derivatives; U.S. Pat. No. 4,683,298 discloses a process for preparing an amino deoxy derivative of a polysaccharide (starch); U.S. Pat. No. 4,435,564 discloses a method for activating HEC (with various amine activators) to facilitate dispersion in a heavy brine solution, to aid in solidification or gelling of such heavy brines.

A water soluble, aminated polymer for use in transfection of cells, with less toxicity than PEI, and greater transfection efficiency than DEAE-dextran would be useful. The present invention provides for compounds comprising carbon polymers and one or more polyamine groups.

### SUMMARY OF THE INVENTION

The present invention relates to compounds comprising carbon polymers and one or more polyamine groups, and methods for their synthesis and use.

Present invention provides a compound comprising a carbon polymer and one or more polyamine groups, the carbon polymer may be selected from the group consisting of hydroxyethylcellulose, dextran, poly(vinyl alcohol) and poly(methy acrylate). Furthermore, the one or more polyamine groups may have the structure of the formula:

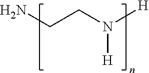

where n is 1 to 10.

The polyamine group may be selected from the group consisting of ethylenediamine, diethylenetriamine, diaminopentane, tris(2-aminoethyl)amine, triethylenetetramine and pentaethylenehexamine and tetraethylenepentamine. Furthermore, the carbon polymer is hydroxyethylcellulose or dextran, and the degree of substitution is greater than one.

The present invention also provides the compound as described above, further comprising one or more than one aliphatic hydrocarbons. The aliphatic hydrocarbon may be saturated or unsaturated. Furthermore, the aliphatic hydrocarbon may be from 2 to 20 carbons in length.

The present invention also provides a composition comprising a carbon polymer and one or more polyamine groups, the carbon polymer may be selected from the group consisting of hydroxyethylcellulose, dextran, poly(vinyl alcohol) and poly(methy acrylate), and a nucleic acid. A pharmaceutical composition comprising the composition and a pharmaceutically acceptable excipient is also provided. A use of the composition for introduction of an exogenous nucleic acid into a cell, and a use of the composition in the manufacture of a medicament for introduction of an exogenous nucleic acid into a cell are also provided. Furthermore, the present invention relates to a kit comprising the compound and instructions for combining the compound with a nucleic acid for transfecting cells with the nucleic acid.

The present invention pertains to a method of transfecting cells with a nucleic acid, comprising contacting cells with the composition comprising a carbon polymer and one or more polyamine groups, and optionally selecting for expression of the nucleic acid. The carbon polymer may be selected from the group consisting of hydroxyethylcellulose, dextran, poly(vinyl alcohol) and poly(methy acrylate). The method may be in vitro, ex vivo or in vivo.

The present invention also provides a compound comprising a carbon polymer and one or more polyamine groups, wherein the carbon polymer is poly(methyl acrylate). A method of preparing the compound with a poly(methyl acrylate) carbon polymer is also provided, the method comprising combining the poly(methyl acrylate) polymer with a polyamine selected from the group consisting of ethylenediamine, diethylenetriamine, diaminopentane, tris(2-aminoethyl)amine and tetraethylenepentamine, and optionally isolating the compound.

The present invention also provides a compound comprising a carbon polymer and one or more polyamine groups, wherein the carbon polymer comprises an hydroxyl group. A method of preparing the compound with a carbon polymer comprising an hydroxyl group is also provided, the method comprising, combining the carbon polymer comprising an hydroxyl group with carbonyldiimidazole to afford an activated oxygen, and reacting the activated oxygen with a polyamine to afford a carbamate linkage between the polymer and polyamine, and optionally isolating the compound.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows (A) the results of a 2-day transfection screen of aminocellulose polymers with 293T cells. Controls, polymer (PEI, aminated cellulose 043002), quantity of polymer and plasmid (gWIZ, gWIZ-GFP) are indicated along the X-axis; the percentage of GPF-positive cells is show along the Y-axis. (B) Toxicity of PEI polymer relative to aminated cellulose 043002 at polymer concentrations of 20 μg/mL—Methylthiazolyldiphenyl-tetrazolium (MTT) absorbance indicated along the Y-axis.

FIG. 2 shows transfection efficiency of PEI and 043002 at 22, 15 and 7.5 μg/mL over 2, 6 and 9 days. Controls, polymer (PEI, aminated cellulose 043002), quantity of polymer and plasmid (gWIZ, gWIZ-GFP) are indicated along the X-axis; the percentage of GPF-positive cells is shown along the Y-axis.

FIG. 3 shows 293T cell counts at 2, 6 and 9 days post transfection (the number of cells as measured by the flow cytometry). Controls, polymers (PEI, aminated cellulose 043002), quantity of polymer and plasmid (gWIZ, gWIZ-GFP) are indicated along the X-axis; Cell counts in Region 1 (i.e., region of interest in flow cytometry corresponding to the viable cell population) is show along the Y-axis.

FIG. 4 shows the results of transfection experiments on bone marrow stromal cells (BMSC) using PEI, or aminocellulose reagents 0616-5, 050201, 050202 and 043002. The percentage of GFP-positive cells is indicated on the Y-axis and transfection reagent on the X-axis, gWIZ plasmid (white bar) and gWIZ-GFP plasmid (black bar)

FIG. 5 shows a comparison of transfection reagents, as assessed by the quantity of green fluorescent protein (GFP) expressed in transfected 293T cells. The percentage of GFP-positive cells is indicated on the X-axis, and the transfection reagent and plasmid transfected is indicated along the Y-axis. PEI is 7.5 μg/mL, all other reagents are 15 μg/mL, “gWIZ” is the empty vector, and “gWIZ-GFP” is the GPF-containing vector. Molecular weight, polymer type and amine groups of the reagents are set out in Table 2.

FIG. 6 shows a Fourier Transform Infrared Spectroscopy (FTIR) spectra of hydroxyethylcellulose (HEC) (dashed line), and HEC modified with diethylenetriamine (DETA) 0616-5 (70 kDa) (circles) and 1204-2 (90 kDa) (solid black line).

FIG. 7 shows a comparison of transfection reagents, as assessed by the quantity of GFP expressed in transfected 293T cells. The percentage of GFP-positive cells is indicated on the X-axis, and the transfection reagent used for plasmid transfected is indicated along the Y-axis. “gWIZ-GFP” is the GPF-containing vector alone without any carrier. “NT”—untransfected cells. All other reagents were complexed with gWIZ-GFP and added to the cells at a concentration of 2 μg/mL (plasmid) and 10 μg/mL (carrier). Reagents are specified in Table 2.

FIG. 8 shows atomic force microscopy images demonstrating the formation of nanoparticles. Nucleic acid (DNA) strands (A) are compacted to nanoparticles (B) in the presence of aminocellulose (reagent 0616-7). Complexes were prepared in 150 mM NaCl with a polymer: nucleic acid ratio of 5.0. Formation of nanoparticles may enable improved update of nucleic acid by cells.

FIG. 9 shows a comparison of transfection reagents, as assessed by the quantity of GFP expressed in transfected 293T cells. gWIZ (white bar), gWIZ-GFP (black bar). Reagents are indicated along the X-axis, the Y-axis illustrates percentage of GFP-positive cells. Reagents are specified in Table 2. All other reagents were complexed with the plasmids and added to the cells at a concentration of 2 μg/mL (plasmid), and 5 μg/mL (PEI) or 10 μg/mL (other carriers).

FIG. 10 shows a comparison of transfection reagents with DEAE-Dextran, as assessed by the quantity of GFP expressed in transfected 293T cells. gWIZ (white bar), gWIZ-GFP (black bar). The percentage of GFP-positive cells is indicated on the Y-axis and the transfection reagent is indicated along the X-axis. Nucleic acids were used as a concentration of 2 μg/mL; “(5)” refers to a polymer:DNA ratio of 5 (10 μg polymer/2 μg DNA), “(10)” refers to a polymer:DNA ratio of 10 (20 μg polymer/2 μg DNA).

FIG. 11 shows a comparison of transfection reagents with PEI and commercial DEAE dextran, as assessed by the quantity of GFP expressed in transfected 293T cells. The percentage of GFP-positive cells is indicated on the Y-axis, and the transfection reagent is indicated along the X-axis, 5 μg/mL (white bar) and 10 μg/mL (black bar) of each reagent are compared. Compounds are specified in Table 2.

FIG. 12a, b shows the results of PCR amplification of Npt sequences in BY2 cells transformed as described. Arrow indicates 484 bp amplicon.

FIG. 13 shows a comparison of the toxicity of transfection reagents with the toxicity of PEI, DEAE dextran and Lipofectamine™ 2000 at two different time points—initial toxicity 2 days post transfection and rebound toxicity at 4 days post transfection. Relative methylthiazolyldiphenyl-tetrazolium (MTT) absorbance indicated along the Y-axis. Compounds are specified in Table 2.

FIG. 14 shows a comparison of transfection reagents using different buffers, as assessed by the quantity of GFP expressed in transfected 293T cells. The percentage of GFP-positive cells is indicated on the Y-axis, and the transfection reagent is indicated along the X-axis. All reagents were complexed with the plasmid and added to the cells at a concentration of 2 μg/mL (plasmid) and 10 μg/mL (carriers). 150 mM NaCl (white bars), 10 mM HEPES-6.8 (black bars) and 10 mM HEPES-4.2 (diagonally patterned bars). Compounds are specified in Table 2.

FIG. 15 shows a comparison of the transfection ability of DETA-dextran (1015-1) using different buffer formulations. The percentage of GFP-positive cells is indicated on the Y-axis, and the buffer formulation used is indicated along the X-axis. DETA-dextran was complexed with the plasmid for 30 minutes in the indicated buffers and added to the cells. After 48 hours, the complexes were removed and GFP expression measured with a fluorescent plate reader.

FIG. 16 shows a comparison of the transfection efficiency of DETA-dextran (1015-1) relative to Escort™ IV and no carrier in different cell lines, as assessed by the quantity of GFP expressed in the cell lines. The percentage of GFP-positive cells is indicated on the Y-axis, and the cell lines are indicated along the X-axis. The plasmid concentration was 1.3 μg/mL in the transfection medium. Cell lines used: MDA 231 (human breast cancer cells), BMSC (rat bone marrow stromal cells), A549 (human lung cancer cells), Vero (African green monkey kidney epithelial cells), HeLa (human ovarian cancer cells) and HepG2 (human hepatocytes). DETA-dextran (black bars), Escort™ IV (grey bars), and no carrier (white bars).

FIG. 17 shows a comparison of transfection reagents at three different concentrations, as assessed by the quantity of GFP expressed in transfected 293T cells. The percentage of GFP-positive cells is indicated on the Y-axis, and the level or concentration of dosing is indicated along the X-axis. All reagents were complexed with the plasmid and added to the cells at three concentrations: high, middle and low concentration. The plasmid concentration in all cases was 1.6 μg/mL. For 1015-1, 1221-1, 0111-1 and 0111-3, the concentrations were 16, 8 and 4 μg/mL for the high, middle and low concentrations, respectively. For PEI, the concentrations were 8, 4 and 2 μg/mL for the high, middle and low concentrations, respectively. Compounds are specified in Table 2. 1015-1 (diagonally patterned bar), PEI (black bar), 1221-1 (horizontal patterned bar), 0111-1 (checkerboard patterned bar), and 0113-1 (white bar).

FIG. 18 shows a comparison of transfection reagents, as assessed by the quantity of GFP expressed in transfected 293T cells. The percentage of GFP-positive cells is indicated on the Y-axis, and the transfection reagent is indicated along the X-axis. “NT” is the non treated control group; “no carrier” is the GFP-containing vector alone without any carrier. All other reagents were complexed with the plasmid and added to the cells at a concentration of 2 μg/mL (plasmid) and 10 μg/mL (carriers). Reagents are specified in Table 2.

### DETAILED DESCRIPTION

The present invention relates generally to compounds comprising carbon polymers and one or more polyamine groups. As described herein, the compounds (aminated polymer) may be combined with an exogenous compound, for example a nucleic acid, to facilitate introduction of the exogenous compound into a cell.

The present invention provides a compound comprising a carbon polymer and one or more than one polyamine group (aminated polymer). Carbon polymers according to various embodiments of the present invention include one or more than one hydroxyl group, or one or more than one ester groups in the monomeric unit of the polymer. Examples of such polymers include but are not limited to hydroxyethylcellulose (HEC), poly(vinyl alcohol) (PVA), poly(methyl acrylate), (PMA), dextran (DEX), pullulan, poly(acrylic acid), poly(methacrylic acid), poly(allyl alcohol) and poly(methyl methacrylate) (PMMA).

Polymers generally may be characterized by their monomeric unit, the molecular weight, the degree of substitution (DS), and in some cases, the identity of side groups. For example, cellulosic polymers (cellulose and HEC) have multiple hydroxyl (—OH) groups that provide reactive centers where chemical modifications may take place. Unmodified cellulose is not soluble in water, however etherification of the hydroxyl groups may be performed to convert the cellulose into HEC, and rendering the polymer soluble.

The degree of substitution (DS) indicates the average number of hydroxyl groups modified per monomer unit—HEC has a maximum of three etherified hydroxyl groups available as reactive centers; therefore, the DS is a maximum of three. For the compounds, compositions and methods described herein, a greater DS is indicative of a theoretical greater density of polyamine groups in the polymer. Linear dextran has a maximum DS of 3, but branched dextrans may demonstrate a DS of less than 3. Molar substitution (MS) indicates the average molar units of a functional group that are present per monomer unit, and may be used to describe functional groups that may be added repeatedly onto a single hydroxyl group (e.g. ethylene oxide, propylene oxide). Some polymers, such as PVA or PMA, may have the average quantity of functional groups described as mol %.

While various methods of production of HEC are known in the art, some may preferentially etherify only one of the possible 3 hydroxyl groups, lending a maximum DS of 1 to the polymer. For the compounds according to various embodiments of the present invention, the substituted group comprises one or more polyamines, as described herein.

Therefore, a polymer with a DS of 1 would have, on average, one polyamine group per monomer; a polymer with a DS of 2 would have, on average, two polyamine groups per monomer, and a polymer with a DS of 3 would have, on average, three polyamine groups per monomer. Elemental analysis may be used to determine the degrees of substitution; methods and formula for this determination are described, for example, by Vaca-Garcia et al., (2001, Cellulose 8(3):225-231).

The molecular weight (MW) of the carbon polymer may be from about 0.5 kDa to about 1000 kDa. For example, the MW of HEC may be from about 1 kDa to about 800 kDa, or any amount therebetween. In some embodiments, the MW may be from about 2 kDa to about 20 kDa, or any amount therebetween, or the MW may be 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, 1000 kDa or any amount therebetween. The MW of PVA may be from about 1 kDa to about 750 kDa. In some embodiments, the MW may be from about 2 kDa to about 40 kDa, or any amount therebetween, or the MW may be 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, 1000 kDa or any amount therebetween. The MW of PMA may be from about 1 kDa to about 750 kDa. In some embodiments, the MW may be from about 2 kDa to about 40 kDa, or any amount therebetween, or the MW may be 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, 1000 kDa or any amount therebetween. The MW of dextran may be from about 1 kDa to about 750 kDa. In some embodiments, the MW may be from about 2 kDa to about 40 kDa, or any amount therebetween, or the MW may be 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, 1000 kDa or any amount. therebetween.

Polyamines according to various embodiments of the present invention may include ethylenediamine (EDA; H₂N(CH₂)₂NH₂), 1,3-diaminopropane H₂N—(CH₂)₃—NH₂, diethylenetriamine (DETA; H₂N(CH₂)₂NH(CH₂)₂NH₂), tetraethylenepentamine (TEPA; H₂N(CH₂)₂NH(CH₂)₂NH(CH₂)₂NH₂), putrescine (H₂N—(CH₂)₄—NH₂, diaminopentane (cadaverine; H₂N—(CH₂)₅—NH₂), spermidine H₂N—((CH₂)₄—NH—)₂—H, spermine H₂N—((CH₂)₄—NH)₃—H and dipropylenetriamine (norspermidine; H₂N(CH₂)₃ NH (CH₂)₃)NH₂), triethylenetetramine and pentaethylenehexamine.

The polyamine group may be described as an ‘ethyleneimine’ unit, having the general structure according to Formula I:

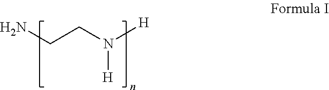

where n=an integer between 1 and 10. For example, which is not be considered limiting, n=1, 2 or 4.

Amines may be generally described as ‘symmetric’ or ‘asymmetric.’ A symmetric amine has the same amine group at either end of the molecule, and provides more reactive groups for coupling with the polymer or crosslinking. Asymmetric amines contain different amine groups at their ends for coupling reactions or crosslinking. Crosslinking may, or may not be a desired effect, depending on the polymer and its intended uses.

TABLE 1
Non-limiting examples of exemplary polyamines according to Formula I

| Chemical Name | Chemical Structure | 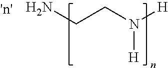 |
|---|---|---|
| Ethylenediamine | 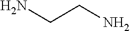 | 1 |
| Diethylenetriamine | 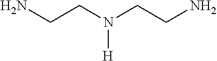 | 2 |
| Tetraethylenepentamine | 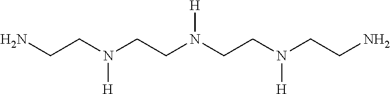 [SMILES: NCCNCCNCCNCCN] | 4 |

In some alternate embodiments, the polyamine may be N-Methylethylenediamine, Propylenediamine, 1,4-Diaminobutane, 3-(Methylamino)propylamine, N,N′-Dimethylethylenediamine, N-Ethylethylenediamine, Diethylenetriamine, 1-Dimethylamino-2-propylamine, 3-(Dimethylamino)-1-propylamine, Cadaverine, N,N,N′-Trimethylethylenediamine, N-Isopropylethylenediamine, N-Propylethylenediamine, 2-(Aminomethyl)-2-methyl-1,3-propanediamine, N-(2-Aminoethyl)-1,3-propanediamine, Bis(dimethylamino)methylsilane, 2,6-Dichloro-p-phenylenediamine, 4,5-Dichloro-o-phenylenediamine, 4-Bromo-1,2-diaminobenzene, 4-Chloro-1,3-diaminobenzene, 4-Chloro-o-phenylenediamine, 4-Fluoro-1,2-phenylenediamine, 4-Fluoro-1,3-diaminobenzene, 2-Nitro-1,4-phenylenediamine, 3-Nitro-1,2-phenylenediamine, 4-Nitro-o-phenylenediamine, 1,2-Phenylenediamine, 1,3-Phenylenediamine, m-Phenylenediamine, o-Phenylenediamine, p-Phenylenediamine, Hexamethylenetetramine, (±)-trans-1,2-Diaminocyclohexane, 1,2-Diaminocyclohexane, cis-1,2-Diaminocyclohexane, trans-1,4-Diaminocyclohexane, 1,5-Diamino-2-methylpentane, 1,6-Diaminohexane, N,N′-Diethylethylenediamine, N,N,N′-Trimethyl-1,3-propanediamine, N,N-Diethylethylenediamine, N,N-Dimethyl-N′-ethylethylenediamine, N-Butylethylenediamine, N-Isopropyl-1,3-propanediamine, N-Propyl-1,3-propanediamine, Bis(3-aminopropyl)amine, Triethylenetetramine, Tris(2-aminoethyl)amine, 3-Bromo-4,5-diaminobenzotrifluoride, 2-(Trifluoromethyl)-1,4-phenylenediamine, 5-(Trifluoromethyl)-1,3-phenylenediamine, 2-Amino-6-fluorobenzylamine, 2,3-Diaminotoluene, 2-Aminobenzylamine, 3,4-Diaminotoluene, purified by sublimation, 4-Aminobenzylamine, 4-Methyl-m-phenylenediamine, 4-Methyl-o-phenylenediamine, N-Methyl-1,2-phenylenediamine, 1,3-Bis(ethylamino)propane, 1,7-Diaminoheptane, 3-(Diethylamino)propylamine, N,N,2,2-Tetramethyl-1,3-propanediamine, N,N-Diethyl-1,3-propanediamine, N,N-Diethyl-N′-methylethylenediamine, 3,3′-Diamino-N-methyldipropylamine, N1-Isopropyldiethylenetriamine, o-Xylylenediamine dihydrochloride, trans-N,N′-Dimethylcyclohexane-1,2-diamine, 1,8-Diaminooctane, 2-(Diisopropylamino)ethylamine, N,N′-Dimethyl-1,6-hexanediamine, N,N,N′-Triethylethylenediamine, N-Hexylethylenediamine, N,N-Diethyldiethylenetriamine, N,N-Dimethyldipropylenetriamine, 1,2-Bis(3-aminopropylamino)ethane, N,N′-Bis(2-aminoethyl)-1,3-propanediamine, Methyl 3,4-diaminobenzoate, 4,5-Dimethyl-1,2-phenylenediamine, 4-(2-Aminoethyl)aniline, m-Xylylenediamine, N,N-Dimethyl-p-phenylenediamine, N-Phenylethylenediamine, Tetraethylenepentamine CP, 2,4,6-Trimethyl-m-phenylenediamine, N-Benzylethylenediamine, N-Tosylethylenediamine, N-Cyclohexyl-1,3-propanediamine, 1,9-Diaminononane, 2,2,4(2,4,4)-Trimethyl-1,6-hexanediamine, 2-Amino-5-diethylaminopentane, N,N-Bis[3-(methylamino)propyl]methylamine, N,N′-Bis(3-aminopropyl)-1,3-propanediamine technical grade, Tris[2-(methylamino)ethyl]amine, 1,4-Diaminonaphthalene, 1,5-Diaminonaphthalene, 1,8-Diaminonaphthalene, 2,3,5,6-Tetramethyl-p-phenylenediamine, N,N,N′,N′-Tetramethyl-p-phenylenediamine, powder, N,N-Diethyl-p-phenylenediamine, 1,8-Diamino-p-menthane, 3-Aminomethyl-3,5,5-trimethylcyclohexylamine, cis-1,8-Diamino-p-menthane, 1,10-Diaminodecane, N,N′-Di-tert-butylethylenediamine, N,N′-Dimethyl-1,8-octanediamine, 1,4-Bis(3-aminopropoxy)butane, 4,7,10-Trioxa-1,13-tridecanediamine, N,N′-Bis(3-aminopropyl)-2-butene-1,4-diamine, N,N′,N″-Trihexyldiethylenetriamine, 4-(Hexadecylamino)benzylamine, 4,4′-(9-Fluorenylidene)dianiline, N,N′-Bis(2-dimethylaminoethyl)-N,N′-dimethyl-9,10-anthracenedimethanamine, N,N′-Bis(2,6-diisopropylphenyl)ethylenediamine, 3,3′-Iminobis(N,N-dimethylpropylamine), Pentaethylenehexamine, N′-Benzyl-N,N-dimethylethylenediamine, 4-tert-Butyl-2,6-diaminoanisole, 3-(Dibutylamino)propylamine, N-(4-Chlorophenyl)-1,2-phenylenediamine, N-Phenyl-o-phenylenediamine, 4,4′-Oxydianiline, 3,3′-Diaminobenzidine, 1,12-Diaminododecane, Bis(hexamethylene)triamine, N,N,N′,N′-Tetraethyldiethylenetriamine, 2,7-Diaminofluorene, 2,7-Diaminofluorene, 3,4′-Diaminodiphenylmethane, 4,4′-Methylenebis(cyclohexylamine), 9,10-Diaminophenanthrene, 4,4′-Ethylenedianiline, meso-1,2-Diphenylethylenediamine, N,N′-Diphenylethylenediamine, N-Methyl-4,4′-methylenedianiline, o-Tolidine technical grade, N,N′-Diphenyl-p-phenylenediamine, 1,1′-Binaphthyl-2,2′-diamine, 2-Amino-N-cyclohexyl-N-methylbenzylamine, N,N′-Dibutyl-1,6-hexanediamine, 2,4,6-Triethyl-1,3,5-benzenetrimethanamine trihydrochloride, 4,4′-Methylenebis(2-methylcyclohexylamine), N,N′,N″-Trimethylbis(hexamethylene)triamine, Tris[2-(isopropylamino)ethyl]amine, N,N′-Dibenzylethylenediamine, triethylenetetramine and pentaethylenehexamine.

TABLE 2
A summary of the transfection agents disclosed herein.

| | | | polyamine or | | additional |
|---|---|---|---|---|---|
| Sample | MW | | amine group | polymer | information |
| 0429-1 | ~40 | kDa | DETA | HEC | |
| 0429-2 | ~40 | kDa | 1,5 | HEC | 0429-2A: |
| | | | diaminopentane | | whole sample |
| | | | | | 0429-2B: |
| | | | | | supernatant of |
| | | | | | centrifuged |
| | | | | | suspension of |
| | | | | | 0429-2A |
| 0429-3 | ~40 | kDa | N-Methyl- | HEC | |
| | | | ethylenediamine | | |
| 0429-4 | ~40 | kDa | tris(2-aminoethyl) | HEC | |
| | | | amine | | |
| 0429-5 | ~40 | kDa | 2-aminoethanol | HEC | |
| 0429-6 | ~40 | kDa | 1,2-dithioethane | HEC | |
| 0429-7 | ~40 | kDa | DETA | HEC | |
| 0429-8 | ~250 | kDa | DETA | HEC | |
| 0429-9 | ~720 | kDa | DETA | HEC | |
| 0429-10 | ~8 | kDa | DETA | HEC | |
| 0429-11 | ~2 | kDa | DETA | HEC | |
| 0429-12 | ~40 | kDa | DETA | HEC | 1.4 wt. % |
| | | | | | etherified with |
| | | | | | tetradecane |
| | | | | | (tetradecyl |
| | | | | | glycidyl ether) |
| 0429-13 | ~40 | kDa | DETA | HEC | 2.2 wt. % |
| | | | | | etherified with |
| | | | | | a tetradecane |
| | | | | | (tetradecyl |
| | | | | | glycidyl ether) |
| 0710 | ~40 | kDa | EDA | PMA | |
| 0713-1 | ~40 | kDa | DETA | PMA | |
| 0713-2 | ~18 | kDa | DETA | PVA | |
| 0713-3 | ~40 | kDa | DETA | PVA | |
| 1007-1 | ~40 | kDa | EDA | PVA | |
| 1007-2 | ~18 | kDa | EDA | PVA | |
| 1015-1 | ~10 | kDa | DETA | dextran | |
| 1015-4 | ~30 | kDa | DETA | birchwood | |
| | | | | hemicellulose | |
| PEI | ~25 | kDa | — | — | poly- |
| | | | | | ethyleneimine |
| 050201 | ~90 | kDa | N,N'-dimethyl- | HEC | |
| | | | ethylenediamine | | |
| 050202 | ~90 | kDa | N,N'-dimethyl- | HEC | Activated |
| | | | ethylenediamine, | | HEC added |
| | | | 1-(3- | | to 1:1 mol |
| | | | aminopropyl) | | mixture of |
| | | | imidazole | | two amines |
| 050203 | ~90 | kDa | N-methyl-1,3- | HEC | |
| | | | diaminopropane | | |
| 041802 | ~90 | kDa | N,N'-dimethyl- | HEC | Activated |
| | | | ethylenediamine, | | HEC added |
| | | | 1-(3-aminopropyl) | | to 1:1 mol |
| | | | imidazole | | mixture of |
| | | | | | two amines |
| 043002 | ~90 | kDa | DETA | HEC | |
| DEAE- | ~500 | kDa | DEAE | dextran | |
| Dex | | | | | |
| 0616-5 | ~70 | kDa | DETA | HEC | |
| 1204-1 | ~30 | kDa | TEPA | HEC | |
| 1204-2 | ~30 | kDa | DETA | HEC | |
| 1204-3 | ~30 | kDa | EDA | HEC | |
| 1204-4 | ~30 | kDa | DETA | HEC | Used 0.5 the |
| | | | | | amount of |
| | | | | | CDI as 1204-2 |
| 1203-2 | ~90 | kDa | EDA | HEC | |
| 1203-3 | ~90 | kDa | TEPA | HEC | |
| 0702-1 | ~90 | kDa | DETA | HEC | |
| 0627-3 | ~89-98 | kDa | DETA | PVA | |
| 1221-1 | ~10 | kDa | DETA | dextran | Used 0.5 the |
| | | | | | of amount |
| | | | | | CDI as 1015-1 |
| 0111-1 | ~10 | kDa | DETA | dextran | Used 0.2 the |
| | | | | | amount of |
| | | | | | CDI as 1015-1 |
| 0111-3 | ~10 | kDa | DETA | dextran | Used 0.65 the |
| | | | | | amount of |
| | | | | | CDI as 1015-1 |
| 0929-2 | ~40 | kDa | EDA | PMA | |
| 0929-4 | ~40 | kDa | DETA | PMA | |

The term “aminocellulose” (and variant spellings) is a general term in the art, and may refer to a variety of cellulose-derived polymers comprising amino groups. Aminocellulose may be prepared by a variety of methods. For example, US 2008/0177021, U.S. Pat. Nos. 4,124,758, 2,136,299, 4,435,564 and 4,683,298 all describe methods to obtain some form of aminated cellulose, but polymer is not defined and may vary.

The compounds according to some embodiments of the present invention are the products of coupling a polyamine with a polymer. For HEC, PVA and DEX, the polyamine coupling is preceded by activation of the polymer by carbonyldiimidazole (CDI). CDI is a mild and selective acylating agent (Staab et al., 1968. Newer Methods Prep. Org. Chem. 5:61-108). For PMA and PMMA polymers, the methyl ester is already suitable for reaction and coupling with the polyamine, therefore a step of activation comprising of CDI is not required. Other etherification or esterification methods, may be used (e.g. directly polymerizing an ethyleneimine monomer (aziridine) onto cellulose). As another example, EDC (1-ethyl-3-(3-dimethylaminopropyl)carbodiimide) or DCC (N,N-‘Dicyclohexylcarbodiimide) may be used to activate —OH groups of the polymer. See, for example, standard references known in the art e.g. Bioconjugate Techniques, 2nd Edition By Greg T. Hermanson, Academic Press, Inc., (2008).

Aminated polymer compounds according to some embodiments of the present invention may further comprise one or more than one aliphatic hydrocarbon, covalently linked via an ether linkage. The aliphatic hydrocarbon may be saturated or unsaturated, and may comprise 2, 3, 4, 5, 6, 7, 8, 9, 10, 11, 12, 13, 14, 15, 16, 17, 18, 19 or 20 carbon units, or any amount therebetween. In some embodiments, the aliphatic hydrocarbon is linear and saturated, and comprises 12, 14, 16 or 18 carbon units. In some embodiments the aliphatic hydrocarbon may be unsaturated.

The aminated polymer compounds of the present invention may be characterized by the quantity of free amino groups on the polymer, using 2,4,6-trinitrobenzen sulfonic acid (TNBS). An exemplary method of such a characterization is described in Siakotos (1967, Lipids 2: 87-88; which is incorporated herein by reference). Briefly, a quantity of the compound is combined with TNBS in a bicarbonate solution and incubated at 37-40° C. Following incubation, an excess of acid is added and absorbance measured at 344 nm. The resulting absorbance is calculated using a standard curve.

The present invention also provides for a method of making a compound comprising a carbon polymer and a polyamine, comprising combining a carbon polymer comprising an hydroxyl group with CDI to produce an activated oxygen, and reacting the activated oxygen with a polyamine to produce a carbamate linkage between the polymer and the polyamine to produce the aminated polymer compound.

The resultant aminated polymer may be used in protocols for transferring a nucleic acid into a cell for the purposes of expression. Such protocols may include (1) clinical protocols where the carrier can be directly applied to subjects (animal or human) in order to modify a host organism directly, (2) ex vivo modification of cells intended for clinical application, where the cells are genetically modified before being applied to the host, and (3) cell culture applications where gene transfer is desired for research and development purposes in general, for example to find out the function of an unknown gene and for expression of protein product from a given gene.

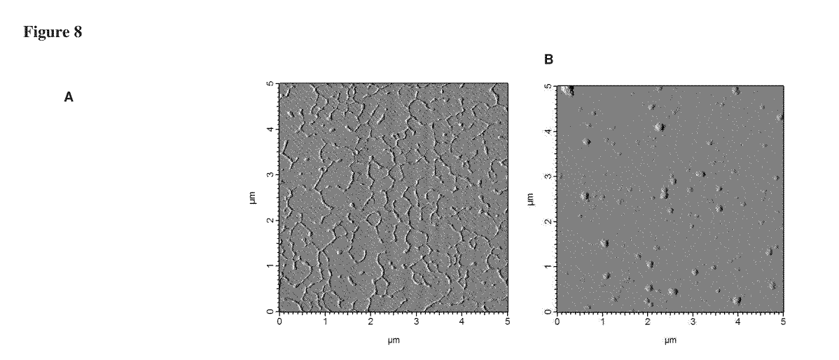

The present invention also provides for a method of transfecting a cell with a nucleic acid. The term “transfection” refers to the introduction of an exogenous compound, preferably a biologically active compound, into a target cell. The exogenous compound may include a macromolecule, nucleic acid, protein, polypeptide, peptide, carbohydrate, lipid, or chemical compound. In some embodiments of the invention a composition comprising an aminated polymer according to the present invention and a nucleic acid may employed to transfect a cell. The aminated polymer compound may be combined with the nucleic acid and allowed to interact for a period of time, for example from about 1 to about 5 minutes, or a longer incubation of about 1 to about 2 hours, or any time therebetween, for example about 15 to about 45 minutes, or any time therebetween, or about 30 minutes. The mass ratio of polymer to nucleic acid may be from about 2 to about 20, or any amount therebetween, for example 3, 4, 5, 6, 7, 8, 9, 10, 11, 12, 13, 14, 15, 16, 17, 18 or 19. In some embodiments the ratio of polymer to nucleic acid is about 5 to about 10. This may result in the formation of nanoparticles, such as those illustrated in FIG. 8.

Transfection may be transient or stable. For many applications, transient transfection, and transient expression of the exogenous nucleic acid is sufficient. The nucleic acid is generally not introduced into the genome, and will eventually be degraded, or diluted as cell division occurs. If stable transfection is desired, the nucleic acid will need to become integrated into the genome of the cell, or else be maintained as an episome as would be known to those of skill in the art. To accomplish this, selective pressure on the cells may need to be maintained, for example resistance to an antibiotic (e.g. geneticin, G418) or a toxin that is added to the media.

The cell to be transfected is contacted by the particles in the presence of culture media (either serum-free, or with serum, the choice may depend on the preferred culture media and the cell type) and allowed to incubate from about 1 hour to about 24 hours, or any time therebetween. Following incubation, the cells are washed to remove the transfection reagents, culture media is replaced and the cells monitored for growth, expression of the nucleic acid, expression of a marker, metabolism, toxicity and the like, as is relevant for the transfected nucleic acid and intended use of the cell.

Particles or nanoparticles formed when the aminated polymer compounds of the present invention are complexed with nucleic acid may be assessed by measurement of size and zeta potential. Size may be assessed by microscopy, such as transmission electron microscopy and atomic force microscopy, or by particle sizing using photon correlation spectroscopy. Zeta potential can be related to the stability of the dispersed particle—a high zeta potential confers stability (a suspension or solution of the particles will resist aggregation).

In some embodiments a delivery system for a biologically active molecule is provided. The biologically active molecule may be a nucleic acid, and the system further comprises one or more of the aminated polymer compounds. The biologically active molecule and the one or more compounds are combined to form a complex, and may be provided to a subject for example, intravenously, topically, or by another mode of administration set out herein or as is known to those skilled in the art (“in vivo”). Alternately, a cell or cells may be removed from the subject and the cells contacted with the complex, transfected, incubated, washed, cultured and screened, and the cells that are successfully transfected, re-administered back to the subject (“ex vivo”). In yet another embodiment, exogenous cells (not from the subject, but may be of the same species, or a different species) are contacted, transfected, incubated, washed, cultured and screened as for ex vivo applications, and then administered to the subject.

The invention further provides compositions for the manufacture of medicaments to treat disorders. Such disorders may be treatable, ameliorable or curable by provision of a biologically active molecule, for example a nucleic acid or other molecule that can be complexed with the aminated polymer compound

Standard reference works setting forth the general principles of cell culture known to those of skill in the art include, for example, Bonifacino et al. (Current Protocols In Cell Biology, John Wiley & Sons, New York, 1998 and Supplements to 2009); Kaufman et al, Eds., (Handbook Of Molecular And Cellular Methods In Biology And Medicine, CRC Press, Boca Raton, 1995); McPherson, Ed; JRW Masters (Animal Cell Culture: A practical approach Oxford University Press, 2000); Elefanty et al. (Current Protocols in Stem Cell Biology, John Wiley & Sons, New York, 2007 and supplements to 2009); Haines et al. (Current Protocols in Human Genetics, John Wiley & Sons, New York, 1994 and supplements to 2009).

The efficiency of a transfection reagent may be assessed by quantifying the expression product of the exogenous nucleic acid. For example, the exogenous nucleic acid may include a selection enzyme that allows the transfected cell to grow and divide in the presence of a drug (e.g. expression of the neo gene confers resistance to the antibiotic G418; expression of the hygromycin B phosphotransferase confers resistance to the antibiotic hygromycin). As another example, a marker may be expressed to allow separation, or visual differentiation between tranfected and untransfected cells (e.g. expression of beta-galactosidase allows visual differentiation of the cells by the blue colour observed when the sugar substrate X-gal is introduced into the media; expression of green fluorescent protein (GFP) allows transfected cells to be distinguished from untransfected cells by the fluorescent signal). GFP as a marker has an additional advantage in that the cells may be sorted using fluorescence activated cell sorting (FACS), and the visualization and sorting process does not damage the cells.

The terms “nucleotide polymer”, “oligonucleotide”, “oligonucleotide polymer”, “oligonucleotide”, “nucleic acid”, “oligomer” or “nucleic acid polymer” are used interchangeably, and refer to polymers comprising at least two nucleotides. A nucleic acid may comprise a single species of DNA monomer, RNA monomer, RNAi, or may comprise two or more species of DNA monomer, or RNA monomers in any combination, including DNA or RNA monomers with modified internucleoside linkages or ‘backbones’. Nucleic acid may be single or double-stranded, for example, a double-stranded nucleic acid molecule may comprise two single-stranded nucleic acids that hybridize through base pairing of complementary bases. The nucleic acid may comprise one or more coding sequences for a polypeptide, enzyme, protein, receptor, hormone or the like. The nucleic acid may comprise a sequence for other motifs or nucleic acid structures of interest including, for example transcription or translational regulatory elements (for example, a promoter, an enhancer, a terminator, one or more signal sequences or the like), vectors, plasmids or the like.

The term “DNA monomer” refers to a deoxyribose sugar bonded to a nitrogenous base, while the term “RNA monomer” refers to a ribose sugar bonded to a nitrogenous base. Examples of DNA monomers that may comprise compositions according to various embodiments of the present invention include, but are not limited to, deoxyadenosine, deoxyguanosine, deoxythymidine, deoxyuridine, deoxycytidine, deoxyinosine and the like. Examples of RNA monomers that may comprise compositions according to various embodiments of the present invention include, but are not limited to, adenosine, guanosine, 5-methyluridine, uridine, cytidine, inosine, and the like. Other DNA or RNA monomers according to various embodiments of the present invention may comprise other nitrogenous bases, as are known in the art.

An internucleoside linkage group refers to a group capable of coupling two nucleosides, as part of an oligonucleotide backbone. Examples of internucleoside linkage groups are described by Praseuth et al (1999, Biochimica et Biophysica Acta 1489:181-206) and Veedu R N et al. (2007, ChemBioChem 8:490-492), both of which are incorporated herein by reference, and include phosphodiester (PO₄—), phosphorothioate (PO3_{S}-), phosphoramidate (N3′-P5′) (PO₃NH) and methylphosphonate (PO₃CH₃), peptidic linkages (“PNA”), locked nucleic acid (“LNA”) and the like. An exogenous nucleic acid, as referenced generally herein, is a nucleic acid produced, or obtained from an external source.

The nucleic acid may be chemically synthesized (see, for example, methods described by Gait, pp. 1-22; Atkinson et al., pp. 35-81; Sproat et al., pp. 83-115; and Wu et al., pp. 135-151, in Oligonucleotide Synthesis: A Practical Approach, M. J. Gait, ed., 1984, IRL Press, Oxford; or Molecular Cloning: a Laboratory Manual 3^{rd} edition. Sambrook and Russell. CSHL Press, Cold Spring Harbour, New York—all of which are herein incorporated by reference), or may be transcribed or copied by an enzymatic process (for example polymerase chain reaction, transcription of a DNA sequence to produce an RNA molecule), or a combination of chemical synthesis and enzymatic processes (for example, extension of a synthetic prime or oligonucleotide by a DNA polymerase). Methods of enzymatic incorporation of LNA nucleosides are described in, for example Veedu R N et al. (2007, Nucleic Acids Symposium 51:29-30), Veedu R N et al. (2007, ChemBioChem 8:490-492), and Veedu et al. (2007, Nucleosides, Nucleotides and Nucleic Acids 26:1207-1210), each of which are incorporated herein by reference.

The sequence of nucleotides comprising a coding sequence or structure of interest may be found, in whole or in part in a publication or a database, for example the GenBank, EMBL, or a similar sequence database. In some embodiments, the nucleic acid transcribed or expressed from the vector (e.g. a DNA vector comprising a nucleic acid sequence of interest is transcribed to provide an RNA molecule within the transfected cell). Nucleic acids may encode a polypeptide that is expressed by the transfected cell, and the polypeptide may be a therapeutic polypeptide. Examples of therapeutic polypeptides include, but are not limited to, cytokines, receptors, enzymes, cofactors, antibodies, fragments of antibodies, transcription factors, binding factors, structural proteins, and the like. The present invention is not to be limited by the nucleic acid being introduced within a cell.

In other embodiments, the nucleic acid may express a marker protein that allows the transfected cell to be distinguished from a non-transfected cell.

Standard reference works setting forth the general principles of recombinant DNA technology known to those of skill in the art include, for example: Ausubel et al. (Current Protocols In Molecular Biology, John Wiley & Sons, New York, 1998 and Supplements to 2001); Sambrook et al, Molecular Cloning: A Laboratory Manual (2d Ed., Cold Spring Harbor Laboratory Press, Plainview, N.Y., 1989); Kaufman et al, Eds. (Handbook Of Molecular And Cellular Methods In Biology And Medicine, CRC Press, Boca Raton, 1995); McPherson, Ed. (Directed Mutagenesis: A Practical Approach, IRL Press, Oxford, 1991).

A target cell may include animal cells, insect cells, plant cells, plant protoplasts, or the like. Non-limiting examples of animal cells include, cell lines obtained from human or non-human mammals, rats, mice, hamsters, rabbits, monkeys, dogs, primates, insects, fish. Examples of cells include but are not limited to human embryonic kidney 293 cells, bone marrow stromal cells (BMSC), insect cells such as Ld652Y, SF9 and SF21, COS cells, MDCK cells, CaCo2, HeLa, stem cells, embryonic stem cells, pluripotent stem cells, induced pluripotent stem cells, Vero cells, LnCAP cells, CHO cells, cancer cell lines derived from humans, or other primary cells derived from patients. Examples of plant cells include tobacco BY-2.

The terms “subject” and “patient” may be used interchangeably. A “subject” refers to an animal, or a mammal, including, but not limited to, a mouse, rat, dog, cat, pig, or primate, including but not limited to a monkey, chimpanzee or human.

Cytotoxicity of the compounds or compositions according to various embodiments may be assessed by any of several methods known in the art. Cell membrane integrity may be assessed as a measure of cell viability or cytotoxic effects. For example, vital dyes such as trypan blue or propidium iodide (normally excluded from a healthy cell) will freely cross the membrane and stain intracellular components. Dead or damaged cells are quantifiable by the blue stain (for trypan blue) or by fluorescence (propidium iodide)—the latter method may be suitable for use with fluorescence activated cell sorting (FACS). In another example, lactate dehydrogenase (LDH) enzyme activity in culture supernatant may be measured—LDH is normally sequestered within a healthy cell, and leaks into the surrounding medium when the cell membrane is compromised. See, for example, Riss T L et al. (Assay Drug Dev Technol 2 (1): 51-62, 2004), Decker T, et al. (J. Immunol. Methods 115 (1): 61-9, 1988), Niles A L, et al. (Anal. Biochem. 366 (2): 197-206, 2007), Fan F et al. (Assay Drug Dev Technol 5 (1): 127-36, 2007; all of which are herein incorporated by reference).

The MTT assay is a standard colorimetric assay that measures the reduction of MTT to formazan (purple indicator). Cytotoxic agents may result in metabolic dysfunction and therefore decreased performance in the assay. The reduction occurs only when mitochondrial reductase is active, and thus the conversion may be used as a measure of viable (living) cells. Note that other viability tests may give different results as various conditions may increase or decrease metabolic activity. When the amount of purple formazan produced by cells treated with an agent is compared with the amount of formazan produced by control cells, the effectiveness of the agent in causing death/changing metabolism of cells is deduced via a dose-response curve.

The amount of a composition administered, where it is administered, the method of administration and the timeframe over which it is administered may all contribute to the observed effect. As an example, a composition may be administered systemically e.g. intravenous administration and have a toxic or undesirable effect, while the same composition administered subcutaneously may not yield the same undesirable effect. In some embodiments, localized stimulation of immune cells in the lymph nodes close to the site of subcutaneous injection may be advantageous, while a systemic immune stimulation may not.

Standard reference works setting forth the general principles of medical physiology and pharmacology known to those of skill in the art include: Fauci et al., Eds., Harrison's Principles Of Internal Medicine, 14th Ed., McGraw-Hill Companies, Inc. (1998).

Pharmaceutical compositions according to various embodiments of the invention may be formulated with any of a variety of physiologically or pharmaceutically acceptable excipients, frequently in an aqueous vehicle such as Water for Injection, Ringer's lactate, isotonic saline or the like. Such excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia (both of which are herein incorporated by reference).

Compositions comprising an antibody or peptide according to various embodiments of the invention may be administered by any of several routes, including, for example and without limitation, intrathecal administration, subcutaneous injection, intraperitoneal injection, intramuscular injection, intravenous injection, epidermal or transdermal administration, mucosal membrane administration, orally, nasally, rectally, topically or vaginally. Alternately, such compositions may be directly injected into a tumor, or a lymph node near a tumor, or into an organ or tissue near a tumor, or an organ or tissue comprising tumor cells. See, for example, Remington—The Science and Practice of Pharmacy, 21^{st} edition. Gennaro et al editors. Lippincott Williams & Wilkins Philadelphia. Carrier formulations may be selected or modified according to the route of administration.

Compositions according to various embodiments of the invention may be applied to epithelial surfaces. Some epithelial surfaces may comprise a mucosal membrane, for example buccal, gingival, nasal, tracheal, bronchial, gastrointestinal, rectal, urethral, vaginal, cervical, uterine and the like. Some epithelial surfaces may comprise keratinized cells, for example, skin, tongue, gingival, palate or the like.

Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006; LL Brunton, editor. McGraw-Hill, New York, or Remington—The Science and Practice of Pharmacy, 21^{st} edition. Gennaro et al editors. Lippincott Williams & Wilkins Philadelphia.

In the context of the present invention, the terms “treatment”, “treating”, “therapeutic use” or “treatment regimen” as used herein may be used interchangeably are meant to encompass prophylactic, palliative, and therapeutic modalities of administration of the compositions of the present invention, and include any and all uses of the presently claimed compounds that remedy a disease state, condition, symptom, sign, or disorder caused by an inflammation-based pathology, infectious disease, allergic response, hyperimmune response, or other disease or disorder to be treated, or which prevents, hinders, retards, or reverses the progression of symptoms, signs, conditions, or disorders associated therewith.

Also provided is an article of manufacture, comprising packaging material and a composition comprising a carbon polymer and one or more polyamine groups and a nucleic acid. The composition includes a physiologically or pharmaceutically acceptable excipient, and the packaging material may include a label which indicates the active ingredients of the composition (e.g. the nucleic acid). The label may further include an intended use of the composition, for example as a therapeutic agent to be used with kits as set out herein.

A kit comprising a composition comprising a carbon polymer and a polyamine as provided herein, along with instructions for use of the composition for introducing a nucleic acid into a cell is provided. The kit may be useful for expression of exogenous nucleic acid in a cell (e.g. as a marker or as a therapeutic), and the instructions may include, for example, dose concentrations, dose intervals, preferred administration methods, methods for screening or testing, or the like.

In another embodiment, a kit for the preparation of a medicament, comprising a composition comprising a carbon polymer and a polyamine as provided herein, along with instructions for its use is provided. The instructions may comprise a series of steps for the preparation of the medicament, the medicament being useful for expressing a nucleic acid in a subject, or in a cell of a subject to whom it is administered. The kit may further comprise instructions for use of the medicament in treatment for the treatment, prevention or amelioration of one or more symptoms of a disease or disorder, and include, for example, dose concentrations, dose intervals, preferred administration methods or the like.

The present invention will be further illustrated in the following examples. However, it is to be understood that these examples are for illustrative purposes only, and should not be used to limit the scope of the present invention in any manner.

### EXPERIMENTAL METHODS AND SYNTHESES

### Materials and Methods

Dimethylacetamide, 1,1′-carbonyldiimidazole, dodecyl and tetradecyl glycidyl ethers, diethylenetriamine (DETA), aminopropyl imidazole, N,N-dimethylethylenediamine, N-methyl-1,3-diaminopropane, ethylenediamine (EDA), tetraethylenepentamine (TEPA), 1,5-diaminopentane, N-Methylethylenediamine, tris(2-aminoethyl)amine, polyvinyl alcohol (cat#s 341584/89-98 kDa, 363138/31-50 kDa, 348406/13-23 kDa), poly(methyl acrylate) (cat#182214), dextran (cat#D9260), birchwood hemicellulose (cat#X0502), and diethylaminoethyl (DEAE)-dextran hydrochloride (cat#D9885) were purchased from Aldrich Chemical Co. Hydroxyethylcellulose (HEC) was obtained from Polysciences Inc. (cat. #05570), and its molecular weight was reduced 3-fold from ˜90,000 to ˜30,000 by sonicating a 35 mg/mL solution of HEC for 120-160 h. Molecular weights were calculated using Polymer Labs Cirrus software, on a Varian pump and RI detector with 3 Polymer Labs aquagel-OH (60, 50, 40) columns using 0.1 M sodium nitrate as the mobile phase. Reported molecular weights are those before modification. FTIR spectra were obtained using a Jasco 4200 spectrometer fitted with a Pike Technologies Miracle ATR accessory. NMR spectra were obtained using a Varian 400 MHz spectrometer. Microelemental analyses were performed by Columbia Analytical Services (Tucson, Ariz., USA).

### Synthesis of Aminocellulose Derivatives

In a typical reaction, a 50 mL round-bottom flask was charged with HEC (0.5 g, ˜5.5 mmol hydroxyl groups) followed by 10 mL (for 30 kDa HEC) or 30 mL (for 90 kDa HEC) dimethylacetamide and stirred overnight or with mild heating to dissolve. Carbonyldiimidazole (0.928 g, 5.7 mmol) was added at once with stirring at 22° C. After 15 minutes, the reaction was then added dropwise to a second 50 mL round-bottom flask containing diethylenetriamine (16.0 g, 155 mmol) with stirring over the course of ˜15 minutes. Dimethylacetamide was added to the stirring diethylenetriamine or starting cellulose solution to decrease viscosity and promote rapid mixing as needed. After 16 hours, the reaction was diluted with isopropanol and precipitated with diethyl ether. The precipitate was washed with acetone/diethyl ether twice, followed by 100% diethyl ether. The product was dissolved in water, dialyzed, and freeze dried to obtain a white solid.

Sample 1204-1 FTIR (ATR) cm⁻¹ 3292, 2934, 2875, 2815, 1707, 1536, 1458, 1254, 1118, 1054, 814, 768. Sample 1204-2 FTIR (ATR) cm⁻¹ 3297, 2926, 2872, 1701, 1538, 1465, 1404, 1255, 1120, 1045, 815, 770. Sample 1204-3 FTIR (ATR) cm⁻¹ 3312, 2931, 2872, 1699, 1533, 1457, 1252, 1116, 1057, 771, 610.

Sample 1204-1 ¹H NMR (D₂O, 400 MHz) δ 4.16 (NCOOCH₁/NCOOCH₂ broad s), 3.93-3.47 (OCH/OCH₂, m), 3.38-2.98 (CONHCH₂, OCH, m), 2.92-2.20 (NCH₂, m). Sample 1204-2 ¹H NMR (D₂O, 400 MHz) δ 4.16 (NCOOCH₁/NCOOCH₂ broad s), 3.94-3.47 (OCH/OCH₂, m), 3.46-2.98 (CONHCH₂, OCH, m), 2.97-2.46 (NCH₂, m). Sample 1204-3 ¹H NMR (D₂O, 400 MHz) δ 4.17 (NCOOCH₁/NCOOCH₂ broad s), 3.82-3.36 (OCH/OCH₂, m), 3.33-2.93 (CONHCH₂, OCH, m), 2.92-2.54 (NCH₂, broad s).

An exemplary synthetic method and polymer structure is illustrated in Scheme 1.

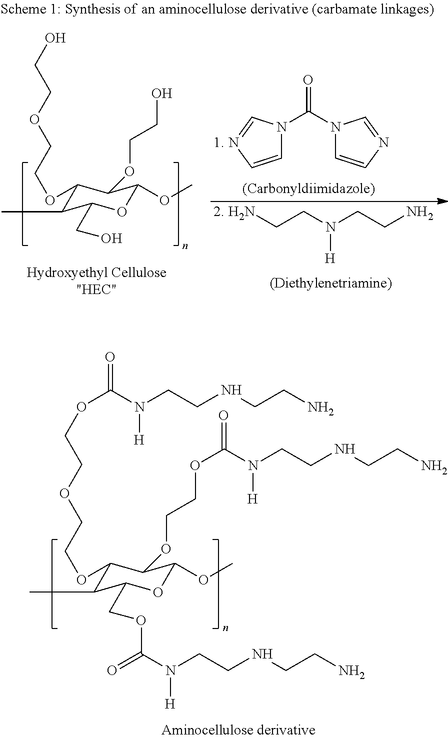

Aminocellulose Derivatives Modified with a Saturated Aliphatic Hydrocarbon

HEC was modified with a saturated aliphatic hydrocarbon as follows. An 8 dram vial was charged with HEC (0.6 g), dodecyl and tetradecyl glycidyl ethers (150 mg, 0.57 mmol or 300 mg, 1.1 mmol), 1.75 g isopropanol, and 1.75 g 1% NaOH with stifling. The reaction was stirred for 5 h at 60° C., precipitated with 25 mL acetone, centrifuged, decanted, and redissolved in 3 mL water/acetone (1:1). The modified HEC was then precipitated a second time with acetone, decanted, and washed two additional times with acetone. The product was dried, dissolved in water, dialyzed, and then freeze dried. This HEC modified with an aliphatic hydrocarbon (12 or 14 carbon chain with an ether-linked epoxide) was then aminated as described in the former method.

Sample 0429-13 FTIR (ATR) cm⁻¹ 3305, 2926, 2870, 1701, 1535, 1458, 1404, 1254, 1118, 1048, 818, 773.

### Synthesis of Dextran Derivative

Dextran was treated in a similar manner to HEC, with dimethyl sulfoxide (DMSO) used as a solvent instead dimethylacetamide. Dextran (25 mg) was dissolved in 0.66 g DMSO and 79 mg carbonyldiimidazole (79 mg, 0.49 mmol) was added with stirring. After 45 minutes, the activated dextran was added dropwise to diethlyenetriamine (1.43 g, 13.9 mmol) with stifling. After 16 hours, the reaction was poured into isopropanol/diethyl ether, dissolved into a small amount of methanol, precipitated again, washed with diethyl ether, dried, dissolved in water, dialyzed, and freeze dried to obtain a white solid.

Sample 1015-1 FTIR (ATR) cm⁻¹ 3277, 2924, 1701, 1535, 1465, 1410, 1258, 1147, 1015, 1015, 775.

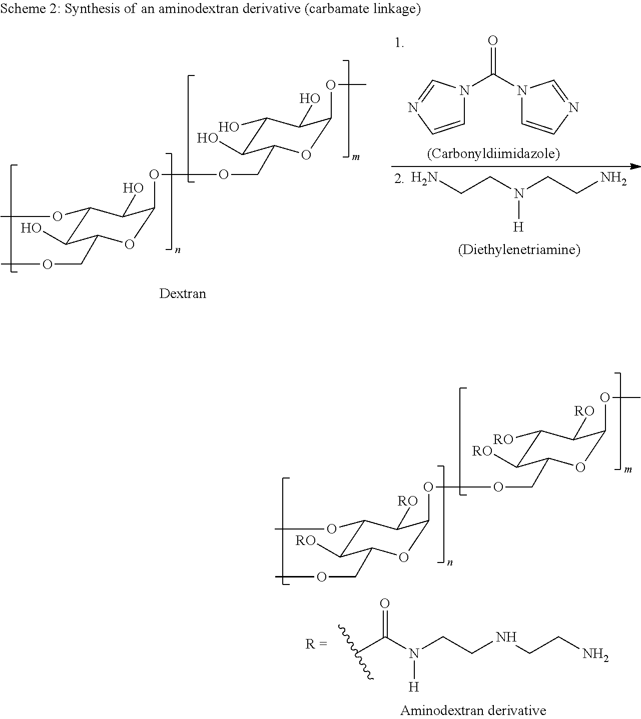

### Synthesis of Hemicellulose Derivatives

Birchwood xylan (25 mg) was dissolved in 1 g dimethylacetamide (DMA), carbonyldiimidazole (45 mg, 0.28 mmol) was added and stirred for 45 minutes. The activated birchwood hemicellulose was added to diethlyenetriamine (0.87 g, 8.4 mmol).

### Synthesis of Modified Poly(Vinyl Alcohol)

The modification of poly(vinyl alcohol) was similar to that of the modified polysaccharides. Poly(vinyl alcohol) (25 mg, 0.57 mmol hydroxyl group) was added to 0.63 g dimethylacetamide, stirred at 100° C. for 30 minutes to dissolve and then cooled to room temperature. To the transparent and colorless solution, carbonyldiimidazole (97 mg, 0.60 mmol) was added with stirring. After 45 minutes, the activated poly(vinyl alcohol) was added dropwise to diethylenetriamine (1.7 g, 16 mmol) or ethylenediamine (1.1 g, 18 mmol) with stirring. After 17 hours, the reaction was poured into isopropanol/diethyl ether, dissolved into a small amount of methanol, precipitated again, and washed with diethyl ether, dried, dissolved in water, dialyzed, and freeze dried to obtain a white solid.

Sample 0713-2 FTIR (ATR) cm⁻¹ 3279, 2931, 2857, 1687, 1541, 1465, 1437, 1261, 1117, 1050, 813, 771. Sample 1007-2 FTIR (ATR) cm⁻¹ 3280, 2931, 2873, 1696, 1523, 1474, 1432, 1260, 1139, 1052, 968, 818, 773.

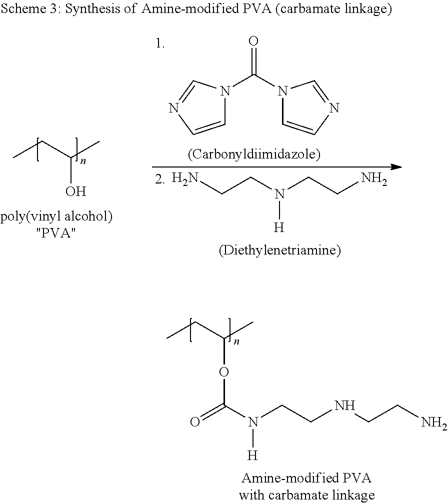

### Synthesis of Modified Poly(Methyl Acrylate)

Excess amine (EDA 0.8 g, 13 mmol or DETA 1.24 g, 12 mmol) was added to a 40 weight % solution of poly(methyl acrylate) in toluene (60 mg, ˜0.28 mmol ester) and stirred for 48 hr at 60° C. with EDA or up to 7 days when using DETA. Reaction time increased with amine length to obtain about 90% conversion of ester to amide. Reactions were added to isopropanol/diethyl ether, dried, dissolved in water, dialyzed, and freeze dried to obtain a white solid.

Sample 0929-2 (prepared in EDA) FTIR (ATR) cm⁻¹ 3278, 3053, 2928, 2867, 1639, 1542, 1438, 1386, 1310, 1241, 1188. Sample 0929-4 FTIR (ATR) cm⁻¹ 3265, 3039, 2932, 2853, 1640, 1551, 1458, 1386, 1297, 1124, 1051.

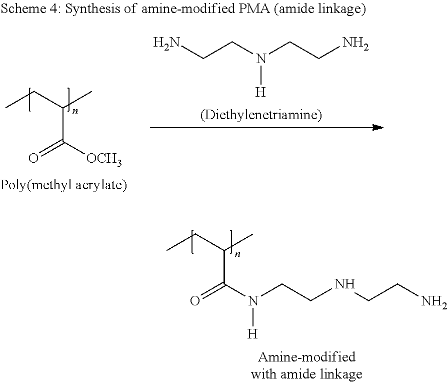

Transfection of Cells

Mammalian cells: The gWIZ transient expression vector (Aldevron) was used to convey the gene of interest (Enhanced Green Fluorescent Protein—EGFP) to the cells. The transfection of cells is typically carried by mixing polymer solutions with a plasmid DNA solution. Human kidney 293 cells (293 cells) or bone marrow stromal cells (BMSC) may be used, grown in Dulbecco's Modified Eagle Medium (DMEM) with 100 U/mL Penicillin, 100 μg/mL Streptomycin, and 10% FBS. The polymer/DNA polyplexes used for transfections were prepared by mixing a desired volume of 0.4 mg/mL DNA plasmid solution (in ddH₂O) carrying a gene of interest (Enhanced Green Fluorescent Protein; pEGFP) (Abbasi et al., 2008 Biomacromolecules 9:1618-1630) with a desired volume of 1 mg/mL polymer solutions (in ddH₂O). The polymer:DNA mass ratios were typically 5 or 10, and exact values are shown in the Figures (also see “Brief Description of the Drawings”). For example, 7.5 μL of 0.4 mg/mL DNA solution may be combined with 15 μL of 1 mg/mL polymer solution to give a mass ratio of 5.0, the volume may be brought to 60 μL typically and then added to the cells in triplicate at 20 μL/well. The total volume is brought to 60 μL with 150 mM NaCl. After a 30 minute incubation at room temperature (allowing for formation of polymer:nucleic acid complexes), the complexes were added to the cells grown on 12-well plates with 0.5 mL medium, and 20 μL of complex solution is added to triplicate wells to give a final plasmid DNA concentration of 2 μg/mL for all experiments. The polymer concentration was variable depending on the experimental purpose. The cells were incubated for 24 hours with the transfection reagents, after which the cells were washed to remove the transfection complexes. At desired time points, the cells were trypsinized for assessment of EGFP expression by flow cytometry. Flow cytometry was performed on a Beckman Coulter Quanta Flow Cytometer, and the cell fluorescence was detected by λₑₓ=485 nm (excitation) and λₑₘ=527 nm (emission) for EGFP expression. The instrument settings were calibrated for each run so as to obtain a background level of EGFP expression of ˜1% for control samples (i.e., cells incubated with pEGFP-N2 alone without any carrier). An aliquot of the cell suspension used for flow cytometry was manually counted with a hemocytometer to obtain total number of cells recovered from the wells.

Plant cells: A seven-day fresh cell suspension of BY2 cells was centrifuged at 1250 rpm for 10 minutes and liquid media was decanted. The pCambia 2301 plasmid (Hajdukiewicz, P. et al., 1994. Plant Mol. Biol. 25 (6):989-994) comprising npt and uidA (beta glucuronidase) genes under control of the CaMV 35S promoter was used for DNA transfer in BY2 cells. PEG6000 mediated transformation was used as a positive control for DNA transfer in BY2 cells (adapted from Protoplast isolation and culture in Arabidopsis. J. Mathur and C. Koncz. Chapter 6. 35-42. In Methods in Molecular Biology: Arabidopsis Protocols Ed. J. M Martinez-Zapater and J. Salinas, Humana Press, Totowa, N.J. 1998). For PEG 6000 transformation, approximately 5 mL centrifuged cells (packed cell volume) were mixed gently with 5 μL DNA and 5 mL of a 40% PEG 6000 solution. Cells were incubated at room temperature for 30 minutes following by three washes in MS basal medium. Cells were either plated on fresh BY2 media for 2 weeks or in liquid BY2 media for two weeks, with weekly sub-culturing. After two weeks kanamycin 50 mg/L cultivation media was used for transformant selection.

For transfection with selected aminated polymers, the media additives and transfection reagents employed were as set out in Table 3. The transfection reagents were vortexed for 2 min to ensure suspension; CaCl₂, spermidine and DNA were added while vortexing continuously for 5 min. 5 mL cultivation medium was added and vortexed for an additional 2 min. The transfection reagent mixture was added to centrifuged cells as above and combined gently. Cells and transfection reagent mixture were transferred to BY solid media and incubated for 48 hours, followed by transfer of the cells to BY selection media (with 50 mg/L kanamycin). Samples for the first GUS staining analyses were taken 24 hours after the subculturing to the selection media. Transfected cells were subcultured on solidified selection media every two weeks, or weekly into liquid BY selection media (50 mg/L kanamycin).

Sonication and vacuum infiltration were used for demonstrating DNA transfer into the cells. 5 mL packed cell volume BY2 cells in BY2 cultivation media were prepared as described above. 100 mL glass flasks with cells and DNA were treated continuously in a sonicator (Branson 5510) at ambient temperature of the water for 2 min. Cells were incubated with DNA on the rotary shaker for 48 hours following transfer to the selection media.

Vacuum infiltration was performed in a similar manner—5 mL packed BY2 cells in media were placed in covered Petri dishes (ø9 cm) for 10 minutes in vacuum chamber). Air was slowly released and cultivation was in 100 mL volume flask for 48 hours, followed by transfer to the selection media.

Histochemical analysis was employed to assess transient and stable GUS expression in the transformed cells. The presence of the Npt gene in proliferated cells on selection media was demonstrated by PCR. Npt primers (npt2a: 5′-CCGCCACACCCAGCCGGCC-3′; npt2s: 5′-CCGACCTGTCCGGTGCCC-3′) amplifying a 484 bp fragment were used. PCR conditions: 95° C., 5 min; 38 cycles—95° C. (1 minute), 62° C. (1 minute), 72° C. (1 minute); followed by a 10 minute extension at 72° C.

TABLE 3
Transfection reagents and additives used for 1204-2 transformation
of BY2 cells. “Uncoated” 1204-2 was used as a control.

| | | | | | | | | | | | | | | | | DNA direct transfer | | | | | |
|---|---|---|---|---|---|---|---|---|---|---|---|---|---|---|---|---|---|---|---|---|---|
| | | | | | | | | | | | | | | | | (lacking mediation) | | | | | |
| | PEG | PEI* | | 1204-2 | | | 1204-2* | | | 1204-2 | | | 1204-2 | | | | | Plus | | Plus Vacuum | |
| Additives | 40% | 5 μg/mL | | 1 μg/mL | | | 5 μg/mL | | | 10 μg/mL | | | 100 μg/mL | | | | | Sonication | | infiltration | |
| CaCl₂ | | | | | | | | | | | | | | | | | | | | | |
| 0M | X | | | X | | | X | | | X | | | X | | | X | | X | | X | |
| 2.5M | | X | | | X | | | X | | | X | | | X | | | X | | X | | X |
| 5.0M | | | X | | | X | | | X | | | X | | | X | | | | | | |
| Spermidine | | | | | | | | | | | | | | | | | | | | | |
| 0M | X | | | X | | | X | | | X | | | X | | | X | | X | | X | |
| 0.1M | | X | | | X | | | X | | | X | | | X | | | X | | X | | X |
| 0.2M | | | X | | | X | | | X | | | X | | | X | | | | | | |

*PEI and 1204-2 were prepared for DNA transfer by different modifications of the protocol for DNA coating on the gold particles.

Maintenance of BY2 Cell Suspension Culture

BY2 cells were maintained in the following media (per litre): 4.3 g MS salts, 30 g sucrose, 0.5 g MES, 4 mL BI-inositol, 3 mL Miller's I, 100 uL 10 mM 2,4-D, pH 5.7. Sterilize by autoclaving. Miller's I-6 g KH₂PO₄ per 100 mL (store at 4° C.); BI-Inositol (2.5×)−(per 200 mL) 0.05 g thiamine, 5 g myo-inositol.

Toxicity Assessment

The cytotoxicity of the polymers was tested on the cells in 48-well flat-bottomed multiwell plates. The cells were seeded with 500 μL tissue culture medium and allowed to attach overnight. The medium used for bone marrow stromal cells was high-glucose DMEM with 100 U/mL Penicillin, 100 μg/mL Streptomycin, 50 μg/mL ascorbic acid and 10% FBS, whereas the medium for 293T cells was low-glucose DMEM with 100 U/mL Penicillin, 100 μg/mL Streptomycin, and 10% FBS. A 1 mg/mL polymer solution was then added to the wells (in triplicate) to give desired polymer concentrations.

The MTT assay (Mosmann, T. 1983. J. Immunological Methods 65(1-2):55-63) was employed to assess the viability of the transfected cells. After 24 hour incubation at 37° C. in a humidified 95/5% air/CO₂ atmosphere, 100 μL of MTT solution (5 mg/mL in Hank's Balanced Salt Solution) was added to each well. After a further ˜2 hour incubation, the medium was removed and 500 μL of DMSO was added to dissolve the MTT crystals formed. The optical density in each well was measured at 570 nm and used as a measure of cell viability. The absorbance of untreated cells was used as a reference control (i.e., 100% cell viability).

### Example 1

### Synthesis of Aminocellulose Derivatives

HEC was treated with carbonyldiimidazole (CDI) as described, to activate the hydroxy groups. Gelation was observed if the reaction was insufficiently mixed, when less than a stoichiometric equivalent of carbonyldiimidazole was used, or with prolonged reaction times of approximately 16 h or more. Without wishing to be limited by theory, gelation may occur as a result of unreacted hydroxy groups crosslinking with activated carbamates to form carbonates, and may be related to polymer length. The gelation was not problematic at the gram scale when using HEC with a molecular weight of 30 kDa. Reactions were allowed to proceed for about 15-120 minutes, and the reaction mixture (comprising HEC+CDI in solvent) was slowly added to a 20-30 fold excess of diethylenetriamine, with rapid mixing, to prevent crosslinking by the symmetric amine.

### Example 2

### Transfection Screening of Aminocellulose Polymers

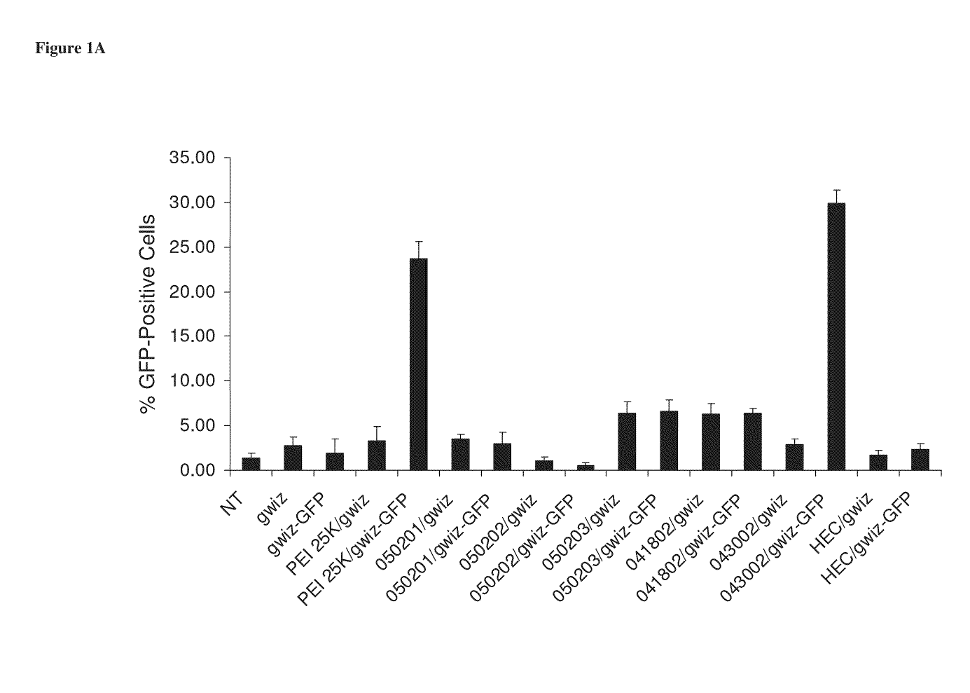

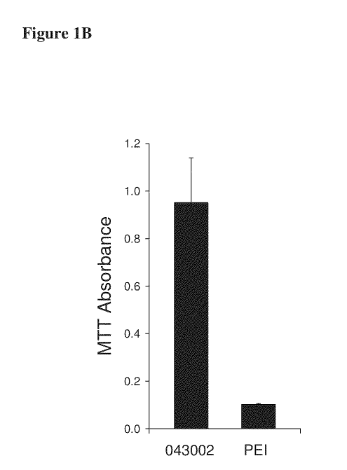

The aminated polymers described in Example 1 were used as transfection reagents in 293T cells. FIG. 1A shows gene expression by cells transfected with PEI and the cellulose derivative containing diethylenetriamine (sample 043002), as well as cellulose derivatives comprising selected asymmetric amines. Asymmetric amines (polymers 050201, 050202, 050203, 041802) demonstrated negligible transfection activity. FIG. 1B shows the relative toxicity of PEI (20 μg/mL final concentration) and 043002 (20 μg/mL final concentration) in 293T cells, using the MTT assay.

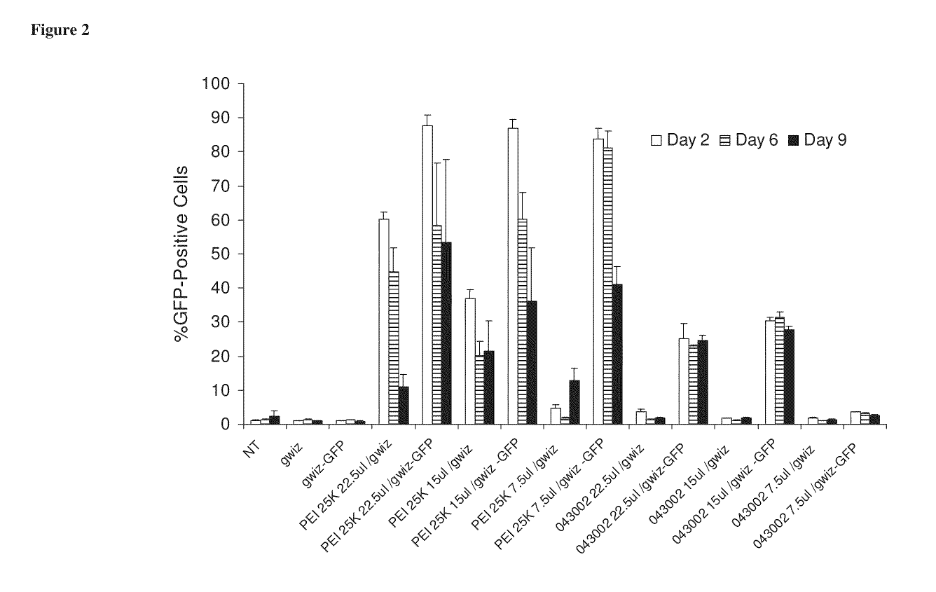

Several differences between PEI and selected aminocellulose derivatives were observed. The gWIZ controls for PEI demonstrated fluorescence—without wishing to be bound by theory, this fluorescence may be autofluorescence resulting from cell disruption due to the toxicity of the PEI transfection reagent, and demonstrated a higher expression than those for 043002. For example, reagent 043002 comprises diethylenetriamine on HEC with a starting MW of 90 kDa (starting MW is the MW of the polymer used in the amination reaction) and cells transfected with this reagent demonstrated less GFP signal overall than PEI at various concentrations (FIG. 2). Without wishing to be bound by theory, this may relate to the efficiency of uptake of the nucleic acid by the cells.

Over the 9 day course of the experiment, GFP expression is generally reduced over time in the PEI-transfected cells, whereas GFP expression is generally steady in the 043002-transfected cells.

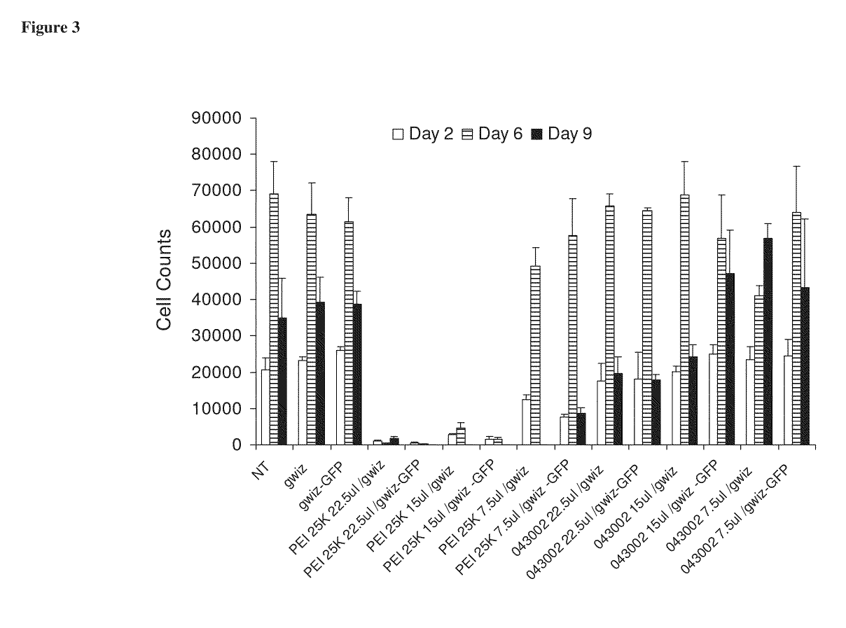

FIG. 3 illustrates the differences in toxicity for the PEI and 043002 reagents at three quantities, as assessed by an MTT assay. PEI demonstrates toxicity for almost all cells at the 22.5 and 15 μg levels, whereas 043002 showed little to no toxicity and yielded cell counts that were equivalent to control (untreated) and plasmid only (no carrier) study groups. Even at the low dose of PEI used (7.5 μg), the cell counts obtained with the 043002 at the end of the experiment (day 9) were greater than PEI-treated cells. Without wishing to be bound by theory, at high concentrations, the cells treated with PEI may not grow due to toxicity effects from the PEI concentration, whereas the cells treated with a similar concentration of cellulosic polymer do not have the same toxicity effect and are able to grow. This suggests that the aminocellulose polymers have an improved compatibility with the cells. Only at low PEI concentration do the cells grow—this may be indicative of a limitation of the use of PEI to the lower concentration ranges.

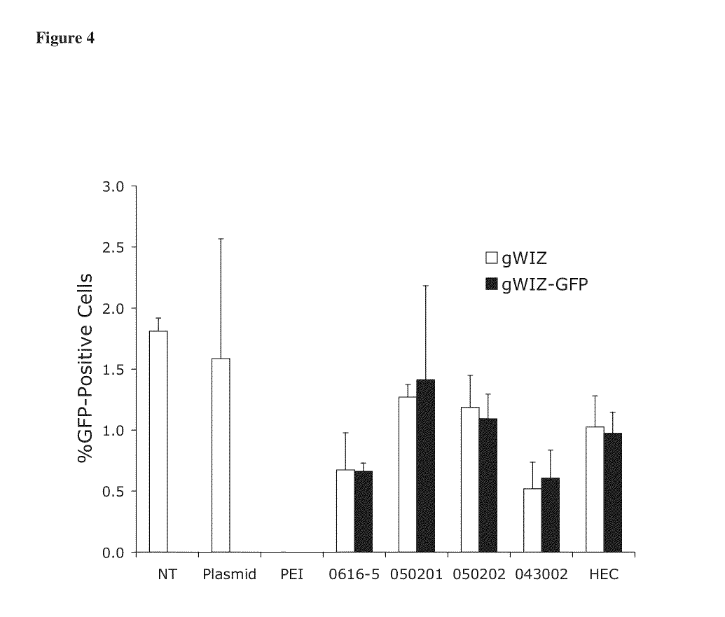

PEI also demonstrates toxicity with bone marrow stromal cells (BMSC). BMSC exposed to PEI were not recoverable, likely due to the high toxicity (no live cells). However, those cells exposed to various aminocellulose derivatives were recoverable, likely because of the low toxicity, albeit in low numbers (FIG. 4). GFP expression above control was not observed.

### Example 3

### Changing the Amine and Molecular Weight

Celluloses modified with ethylenediamine (EDA) and tetraethylenepentamine

(TEPA) were also produced. These amines, ethylenediamine and tetraethylenepentamine, can be seen in Table 1. EDA, DETA and TEPA contain 1, 2, and 4 ethyleneimine units per molecule, respectively.

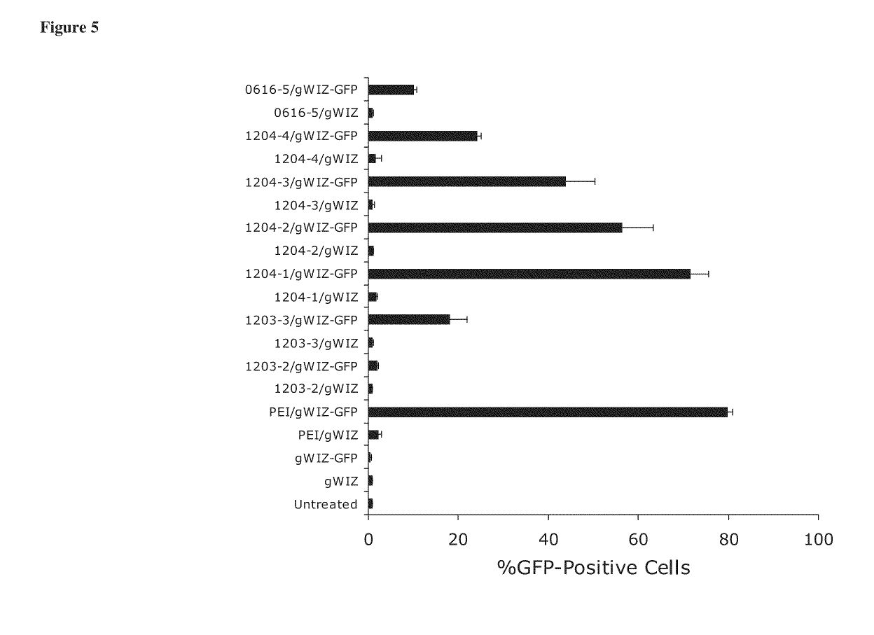

FIG. 5 shows the results of transfection experiments using reagents comprising EDA, DETA or TEPA. As the amine length increases, a higher percentage of cells demonstrate GFP expression. This positive correlation between amine size and transfection efficiency (as measured by GFP expression) arises likely due to the concentration of amine, for a given polymer concentration, increases as amine size is increased. Polymer concentrations are the same for all entries in FIG. 5—7.5 ug/mL for PEI and 15 μg/mL for all cellulose derivatives. For example, the concentration of amine groups for reagent 1204-2 (comprising DETA) is greater than 1204-3 (comprising EDA), and a higher expression of GFP is observed in the 1204-2 treated cells. A similar trend is seen for 1204-1 and 1204-2.

Reagents 1204-4 and 1204-2 are both about 30 kDa and comprise DETA, however, 1204-4 has 37% less diethylenetriamine substitution than reagent 1204-2. This was achieved by using 50% less CDI for the synthesis of 1204-4, thereby leaving more hydroxyl groups in the final product. The transfection efficiency of 1204-4 is reduced by 43% as a result.

TABLE 4
Elemental Analysis:

| Sample | % C | % H | % N | DS |
|---|---|---|---|---|
| 1203-2 | 43.93 | 7.11 | 6.78 | 0.8 |
| 1203-3 | 49.06 | 8.67 | 17.68 | 1.5 |
| 1204-1 | 48.14 | 8.09 | 18.05 | 1.6 |
| 1204-2 | 44.21 | 8.46 | 15.53 | 1.9 |
| 1204-3 | 43.66 | 6.88 | 12.57 | 2.0 |
| 1204-4 | 46.45 | 6.80 | 12.07 | 1.2 |

Upon further analysis, another trend can be found. The nitrogen contents of 1204-3 and 1204-4 are similar, yet 1204-3 performs better (FIG. 5, Table 2). Sample 1204-3 has close to double (1.7 times) the substitution of 1204-4, but with a smaller amine (ETA instead of DETA). Consequently, 1204-3 contains 0.56 fewer unreacted hydroxyl groups as 1204-4 on a weight-to-weight basis. The converse is observed when comparing 1204-3 and 1204-1 with 1204-1 performing better than 1204-3. Reagent 1204-3 has 0.77 fewer hydroxyl groups on a weight-to-weight basis, yet it does not demonstrate as high of a transfection efficiency as 1204-1 which contains 1.4 times more nitrogen. Without wishing to be limited by theory, the unreacted hydroxyl group negatively affects transfection efficiencies, but the negative effects can be circumvented by using a longer polyamine to increase nitrogen content.

DS may be determined from the elemental analysis (Table 4). DS was calculated based on % N, assuming a MS (molar substitution) of 2.5 for the HEC.

One possible explanation (again, without wishing to be bound by theory) is that the HEC hydroxyl groups interact with amino groups, and hinder their complexation with DNA. The data presented suggests that the differences in transfection efficiency differences between reagents 1204-1, 1204-2, 1204-4 and 1204-4 may arise from amine composition and substitution, and further suggests that the repeating ethyleneimine structure, CH₂CH₂NH, may have a role in transfection and/or complexation of the nucleic acid, which in turn may make for a more efficient transfection.

Other performance differences between the reagents presented in FIG. 5 may be attributed to molecular weight, solubility or sample reaction conditions, or a combination thereof. During synthesis, sample 1203-2 began to thicken/gel within 20 minutes of adding carbonyldiimidazole. Consequently, only a portion (about one half to about one third) of the 0.5 g of HEC was added to the diamine. The transfection from 1203-2 is poor when compared to 1204-3, its lower molecular weight analog. Interestingly, the elemental analysis of 1203-2 shows its nitrogen content is only 54% to that of 1204-3. The FTIR shows 54% smaller carbamate stretch (1700 cm⁻¹) as well. This suggests that the dissolution and/or crosslinking of 1203-2 may have hindered its activation and amination.

Sample 1203-3 had similar gelling problems within 15 minutes following the addition of carbonyldiimidazole. The water soluble component was separated from the gel component using centrifugation followed by a 5 um filtration of the supernatant. The 1203-3 filtrate was then used for the transfection experiment and was outperformed by similar but lower molecular weight reagent 1204-1. FTIR analysis (not shown) indicated that this filtrate had a higher amine group concentration than that of the reagent before filtration.

A 25 mg reaction was performed the same day as 1203-2 and 1203-3, using the exact same reagents. However, this small scale solution showed no signs of gelling or viscosity increase even after several hours of stirring with carbonyldiimidazole. Its concentration was 3.3%, twice that of 1203-2.

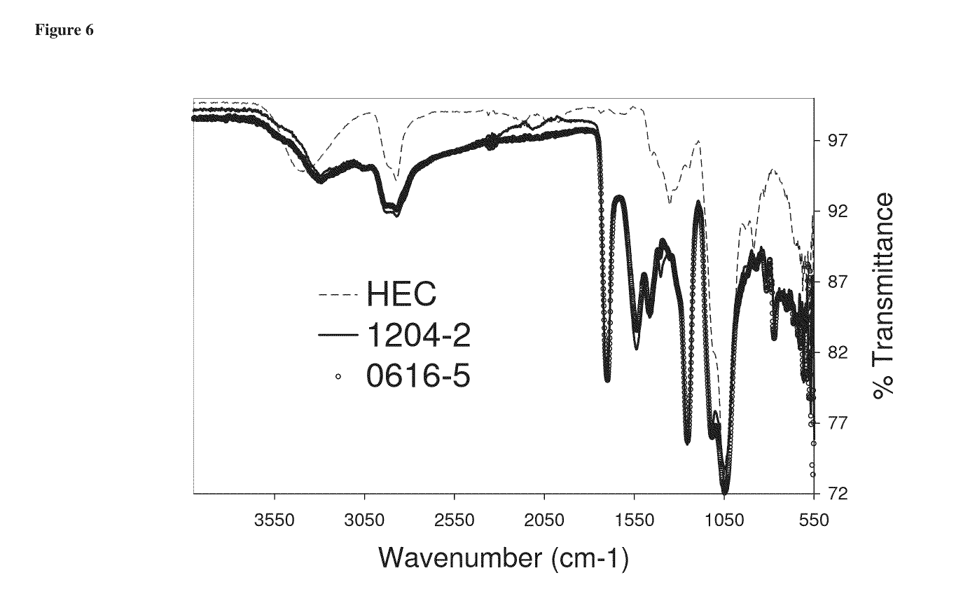

Reagent 0616-5 (comprising DETA) was sonicated for a minor molecular weight reduction to 70 kDa. It was prepared at the 50 mg scale, whereas all other samples in FIG. 5 were prepared with 0.5 g HEC as starting material. However, reagent 1204-2 (comprising DETA and 30 kDa in size), which has the same amine, had almost six-fold greater transfection efficiency than 0616-5. The modification level appears similar, as indicated by similar carbamate stretches (1700 cm⁻¹) in the FTIR (FIG. 6). This, in combination with the transfection data, suggests that the molecular weight may cause reduced transfection efficiency. The larger molecular weight HEC appears to have a greater propensity to crosslink, which may result in a less soluble aminocellulose product, lower substitution, and/or less accessible amine groups. Without wishing to be bound by theory, as aminocellulose needs to be in solution to interact with DNA, insoluble aminocellulose decreases the actual solution concentration resulting in lower transfection efficiencies.

### Example 4

### Efficiency of Alternate Amines, and Polymers

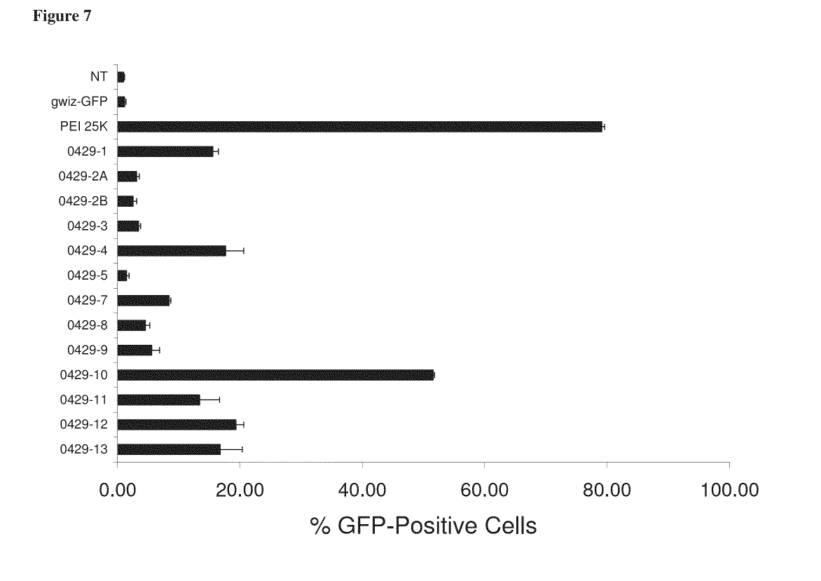

Previous experiments suggested the importance of an ethyleneimine unit as part of the amine group of the transfection reagents tested. FIG. 7 illustrates the results of further investigation of the amine or polyamine group, molecular weight of the cellulose polymer and the effect of hydrophobic modification on nucleic acid delivery to the transfected cells. Table 2 sets out further information on the polymers tested. Reagent 0429-1 (DETA, ˜40 kDa and comparable to 0616-7 and 1204-2) was the standard for comparison. Reagent 0429-2 (1,5 pentanediamine, ˜40 kDa) was purified by two different methods. Reagent 0429-2A is the complete dialyzed material-just like other samples, whereas 0429-2B is the supernatant after sub-sampling 0429-2A, centrifuging, and freeze-drying. Both products of 0429-2 demonstrated lower transfection efficiency (as measured by GFP expression), suggesting that a terminal amine is not sufficient (of itself) for an efficient transfection reagent. Reagent 0429-3 demonstrated transfection efficiency similar to that of the 0429-2 reagents, further suggesting the importance of the native ethyleneimine structure. The performance of reagent 0429-5 suggests the carbamate and the terminal hydroxy group of ethanolamine are not functional; IR spectroscopy indicated that little to no primary amine was present. The transfection ability of reagent 0429-4 is similar to that of 0429-1—suggesting that a branched ethyleneimine is advantageous. The synthesis of 0429-4 required more amine due to the tri-functional, rather than di-functional, amine to prevent crosslinking. During synthesis excess amine used was 75 mol equivalents instead of 30 mol equivalents. The 0429-6 reagent was synthesized, but was not soluble in water, and therefore not tested as a transfection reagent.

Reagent 0429-7 was prepared by quickly adding as a single aliquot the amine (DETA) to the solution of activated HEC (instead of dropwise). However, the final product had less solubility than sample 0429-1, suggesting that one-step addition of a symmetric diamine is not enough to prevent crosslinking, and that controlled addition of activated HEC to the amine may be a preferable method (relative concentration of amine to activated hydroxyl groups is kept high, and thus crosslinking is minimized). Sample 0429-9 was prepared in more dilute conditions than 0429-8 to reduce solution viscosity of the higher molecular weight HEC. This may have lowered crosslinking for 0429-9 relative to 0429-8, however both reagents exhibited similar transfection efficiency.

Reagent 0429-10 demonstrated the highest transfection efficiency of the HEC polymers' results shown in FIG. 7. The improved transfection efficiency observed with the 8 kDa polymer may be a result of one or more characteristics. Without wishing to be limited by theory, these may include one or more of improved solubility, formation of DNA-polymer particles with improved cellular uptake, more efficient nucleic acid complex formation or the like. Further reduction in polymer size to 2 kDa (reagent 0429-11) did not exhibit a further increase in transfection efficiency. Again, without wishing to be limited by theory, this may be a result of one or more of less stable DNA-polymer particles, DNA-polymer particles which are poorly taken up by cells, or the like. Reagents 0429-12 and -13 modified with an aliphatic group demonstrated transfection efficiencies similar to that of 0429-1.

Atomic force microscopy studies (FIG. 8) illustrated the capability of aminocellulose (reagent 0616-7 in 150 mM NaCl, with a polymer:DNA ration of 5:1) to compact DNA into nanoparticles suitable for cell uptake.

### Example 5

### Polymer Backbone with Amide or Carbamate Linkage

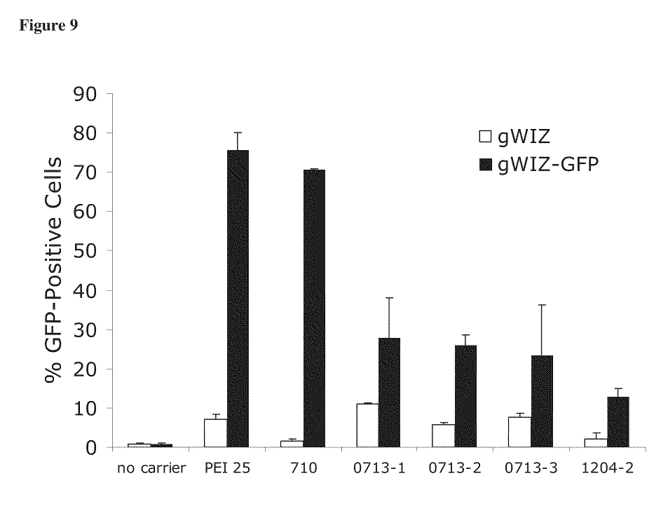

Poly(methyl acrylate) was used to test for differences between amide and carbamate linkages. As described in the methods section, the methyl ester functional group of the PMA substrate was converted to an amide using excess diamine (EDA or DETA). PEI, reagent 1204-2 (HEC with DETA, ˜30 kDa) were included for comparison. The amide of 0713-1 (PMA with EDA, ˜40 kDa) performed similarly to the carbamate in samples 0713-2 (PVA with DETA, ˜18 kDa) and 0713-2 (PVA with DETA, ˜40 kDa) (FIG. 9). Table 2 provides a brief description of the backbone, molecular weight and amine group for the polymers tested.

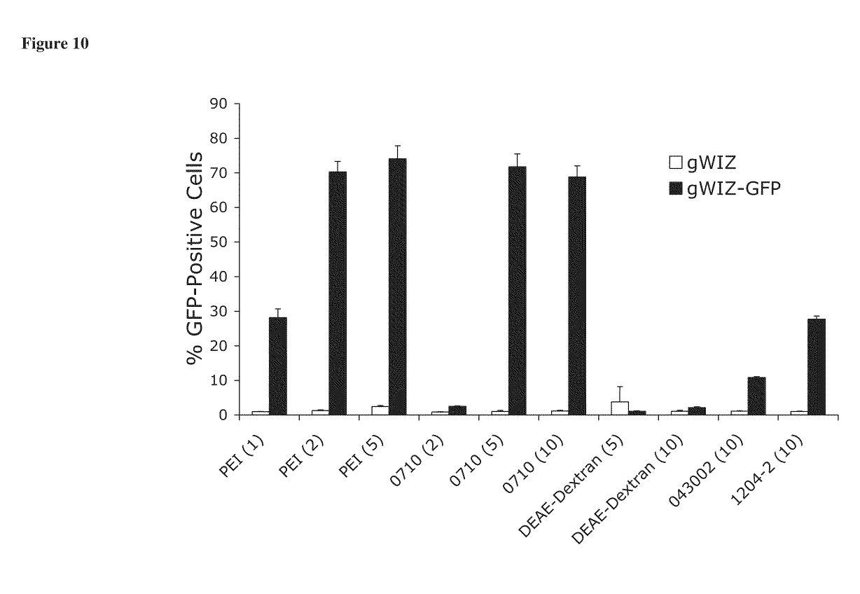

It was expected that the smaller amine, ethylenediamine, on sample 0710 would perform slightly worse than diethylenetriamine on sample 0713-1 because there is less amine (active component) per unit of mass for sample 0710. However, the opposite was found and sample 0710 showed transfection efficiencies similar to that of PEI (FIGS. 9 and 10).

Analogs of 0713-2 and 0713-3 were made using the smallest amine (ethylenediamine). Although the new reagents 1007-1 and 1007-2 (PVA analogues) were not directly compared to the polyvinyl alcohol analogs functionalized with diethlyenetriamine (0713-2 and 0713-3), they showed over a 3-fold increase in % GFP positive cells when compared to 1204-2 and comparable transfection efficiencies to 0710. The evolving trend for these synthetic backbone polymers seems to be higher transfection efficiencies for modification with ethylenediamine over the larger diethylenetriamine.

### Example 6

### Transfection with DEAE-Dextran

Diethylaminoethyl (DEAE)-dextran is a commercial, aminated polysaccharide that has been suggested as useful for transfection of cells. FIG. 10 shows a comparison of DEAE-dextran (avg MW 500 kDa) with selected aminated polymers described herein. FIG. 10 shows DEAE-dextran results with previously discussed samples. Relative to other polymers tested, DEAE-dextran did not demonstrate significant transfection efficiency.

### Example 7

### Using Other Polysaccharides

Hemicellulose and dextran were aminated with DETA and tested for transfection efficiency. Table 2 provides a brief description of the backbone, molecular weight and amine group for the polymers tested; amination was performed as described for HEC—dextran and hemicellulose from birchwood were activated with carbonyldiimidazole and modified with diethylenetriamine. FTIR results showed a greater extent of amination for dextran.

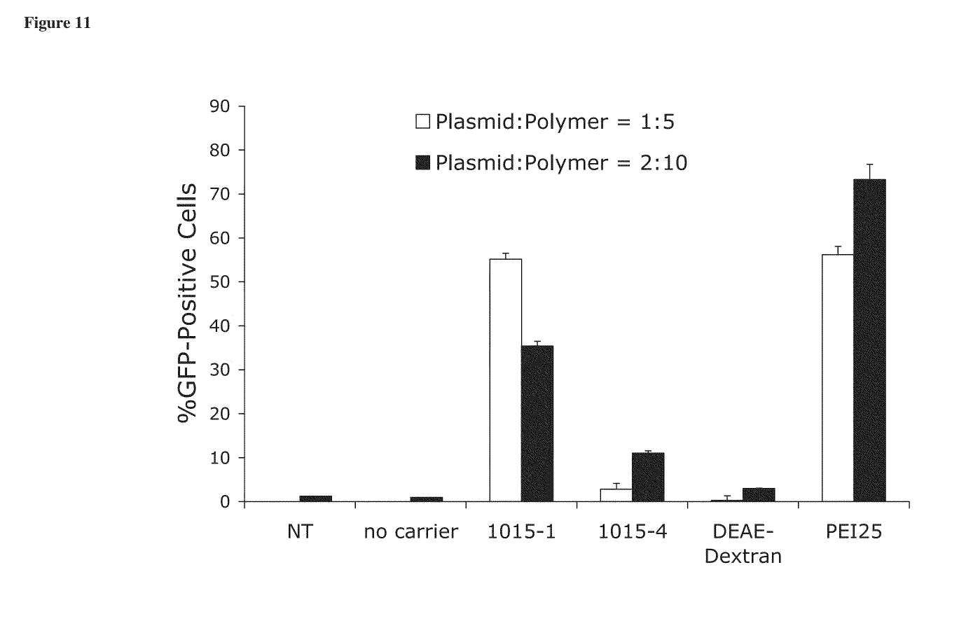

Dextran showed a higher transfection efficiency relative to the birchwood hemicellulose derivative (FIG. 11). These results support the hypothesis that any polysaccharide which can be modified in a polar organic solvent could yield a product with transfection activity.

### Example 8

### Transfection of BY2 Cells

Transient GUS positive results were observed in the variants highlighted and bolded in Table 5. Cells transfected with PEG 6000, PEI or 10 or 100 μg/mL 1204-2 polymer, with stable GUS expression observed with PEG, PEI and 1204-2—100 μg/mL transfection experiments. BY2 cells were subcultured every week on liquid or bi-weekly solid selection media and fast dividing cells were collected from solid BY2 cultivation media, supplemented with 50 mg/L kanamycin.

Cells transfected with 100 μg/mL of polymer 1204-2 exhibited the strongest GUS expression (presence of blue pigment) in the supernatant; transfection with PEG, PEI or 10 μg/mL polymer 1204-2 exhibited GUS expression, but less than that of the 100 μg/mL transfection.

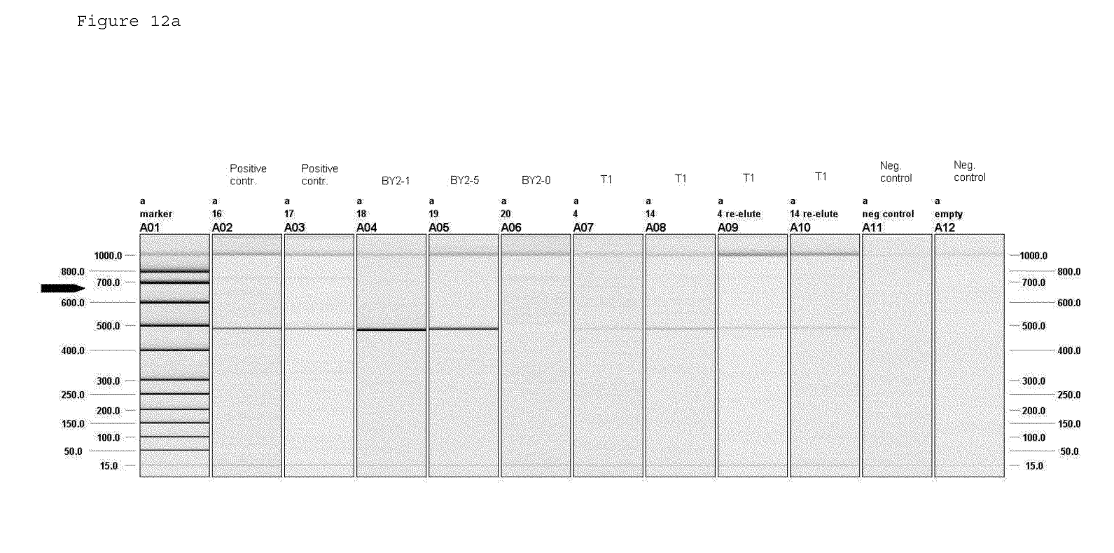

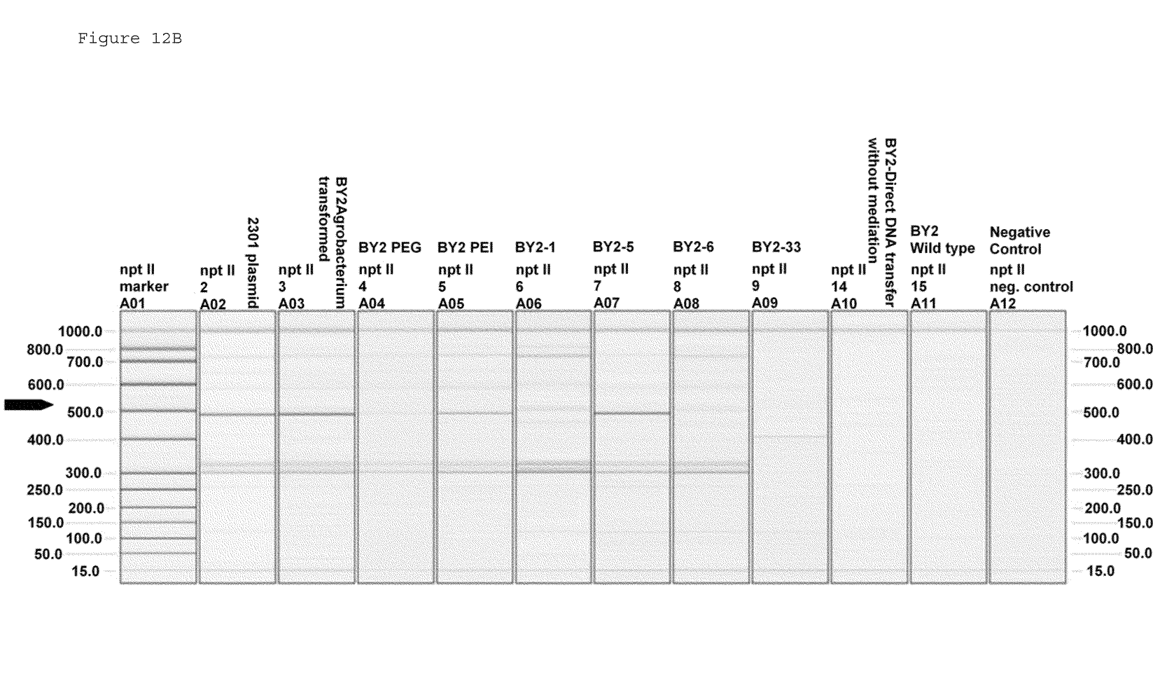

The presence of NPG coding sequence was verified by PCR as described (FIGS. 12 a, b). Two replicates of each one the growing cultures were used to demonstrate the transgenic nature of the proliferated cells.

TABLE 5
GUS positive reaction was observed in highlighted and bolded variants

| | | | | | | | | | | | | | | | | DNA direct | | | | | |
|---|---|---|---|---|---|---|---|---|---|---|---|---|---|---|---|---|---|---|---|---|---|
| | | | | | | | | | | | | | | | | transfer (lacking mediation) | | | | | |
| | PEG | PEI | | 1204-2 | | | 1204-2 | | | 1204-2 | | | 1204-2 | | | | | Plus | | Plus Vacuum | |
| Additives | 40% | 5 μg/mL | | 1 μg/mL | | | 5 μg/mL | | | 10 μg/mL | | | 100 μg/mL | | | | | Sonication | | infiltration | |
| CaCl₂ | | | | | | | | | | | | | | | | | | | | | |
| 0M | 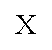 | | | X | | | X | | | X | | | X | | | X | | X | | X | |
| 2.5M | | 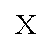 | | | X | | | X | | | 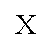 | | | 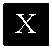 | | | X | | X | | X |
| 5.0M | | | X | | | X | | | X | | | X | | | X | | | | | | |
| Spermidine | | | | | | | | | | | | | | | | | | | | | |
| 0M | 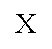 | | | X | | | X | | | X | | | X | | | X | | X | | X | |
| 0.1M | | 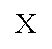 | | | X | | | X | | | 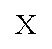 | | | 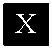 | | | X | | X | | X |
| 0.2M | | | X | | | X | | | X | | | X | | | X | | | | | | |

### Example 9

### Toxicity of Different Compounds

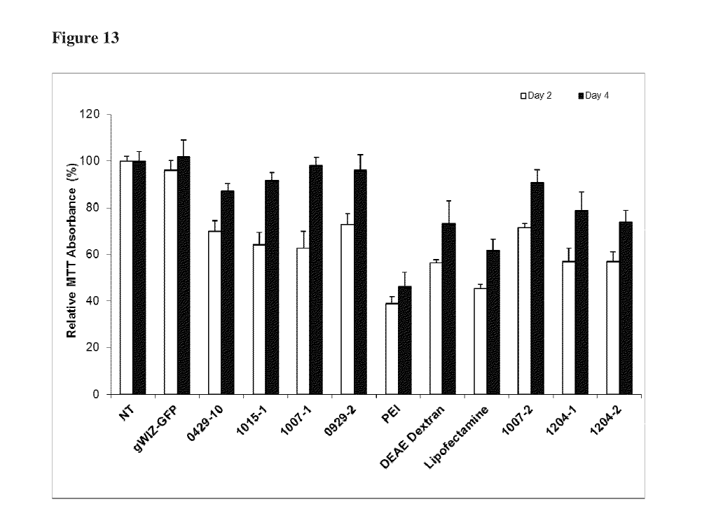

Toxicity of several compounds of the present invention (0429-10; 1015-1; 1007-1; 0929-2; see Table 2) was tested. The results are shown in FIG. 13. Known commercial compounds, such as PEI, DEAE-Dextran and Lipofectamine™ 2000 (Invitrogen) are also provided for comparison. The compounds were tested at two different time points (2 days post transfection and 4 days post transfection), and assessed using the MTT assay described above (Mosmann, T. 1983. J. Immunological Methods 65(1-2):55-63). The different compounds were complexed with the gWIZ-GFP vector plasmid and added to the cells (e.g. 293T cells) t a concentration of 2 μg/mL (for the gWIZ-GFP plasmid) and 10 μg/mL (for the compound carrier). The non-treatment control (“NT”) was designated as 100% viable and the transfection reagents were compared to the non-treatment control.

The different compounds shown in FIG. 13 showed low toxicity in comparison to commercial compounds. The commercial compounds known in the art demonstrated greater toxicity at both time points compared to the compounds of the present invention. PEI shows the highest toxicity at two days and four days post-transfection. The toxicity of the compounds decreased for all the compounds by the second time point (i.e., four days post transfection), yielding a higher cell count for all the compounds four days post transfection compared to two days post transfection. Samples 0429-10, 0929-2 and 1007-2 demonstrated the lowest toxicity two days post transfection, and samples 1007-1 and 0929-2 showed the lowest toxicity four days post transfection.

### Example 10

### Affect of the Buffer on Transfection Efficiency

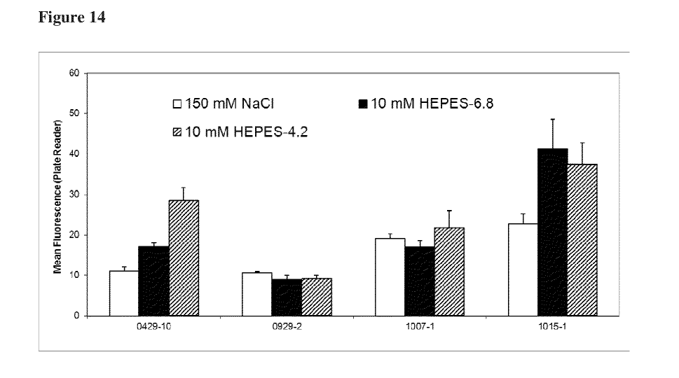

Compounds of the present invention were further tested for their transfection efficiency in 293T cells when different buffers were used during the transfection protocol (see “Transfection of Cells”, above). FIG. 14 shows the transfection results of selected aminated polymers described herein using three different buffers: 150 mM NaCl (white bars), 10 mM HEPES-6.8 (black bars), and 10 mM HEPES-4.2 (diagonal patterned bars). The different compounds were complexed with gWIX-GFP plasmid and added to the 293T cells at a concentration of 2 μg/mL (gWIX-GFP plasmid) and 10 μg/mL (carrier/compound).

For samples 0429-10 (DETA-HEC) and 1007-1 (EDA-PVA), the use of 10 mM HEPES-4.2 buffer showed the highest transfection efficiency over the other buffers for those samples. 10 mM HEPES-6.8 buffer with sample 1015-1 (DETA-dextran) was also an effective combination and produced the highest transfection efficiency in this analysis as evidenced by the quantity of GFP expressed in the transfected 293T cells. For all three different buffer conditions, sample 1015-1 (DETA-dextran) exhibited good transfection efficiency when compared to the other aminated polymers tested. Sample 0929-2 (PMA) showed the lowest transfection efficiency in 293T cells, with 10 mM HEPES-6.8. These results suggest that a range of buffers may be used as all aminated polymers tested retained transfection ability in all buffer conditions tested. However, it may be beneficial to sample various buffers with selected aminated polymers during transfection as the buffer may have an effect on the efficiency of transfection.

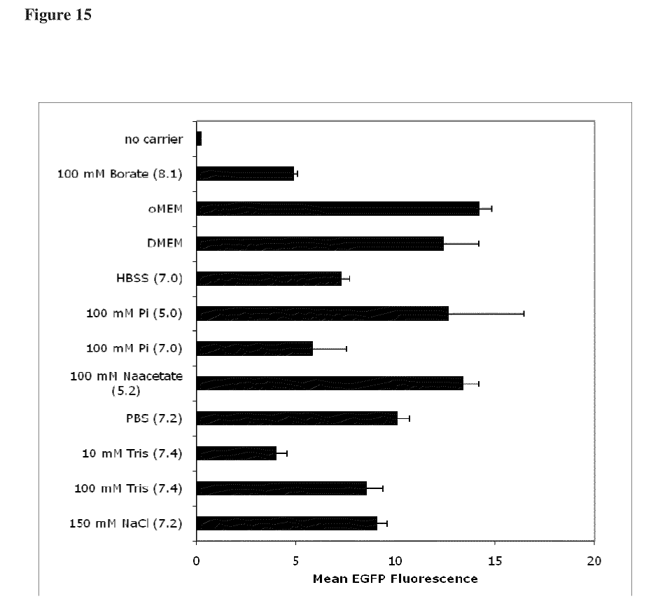

The transfection efficiency of sample 1015-1 using several different buffers were further tested (see FIG. 15). The carrier 1015-1 was complexed with gWIZ-GFP plasmid for 30 minutes in the buffers indicated in FIG. 15 and then added to the cells (e.g. 293T cells). Forty-eight hours post transfection, GFP expression was measured using a fluorescent plate reader. All complexes prepared with sample 1015-1 (DETA-dextran) retained their transfection ability in all buffer conditions tested, as indicated by significantly higher GFP fluorescence as compared to the no carrier control (i.e., gWIZ-GFP plasmid only). There was some variability of the aminated dextran using different buffer conditions, with the use of oMEM, DMEM, 100 mM Pi (5.0) and 100 mM Na acetate (5.2) demonstrating the highest transfection efficiency for sample 1015-1.

### Example 11

### Transfection Efficiency of Dextran in Different Cell Lines

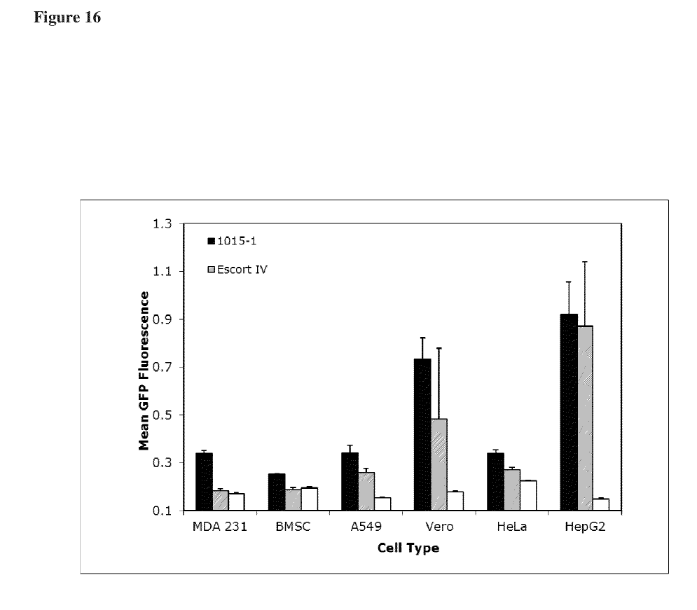

Dextran aminated with DETA (sample 1015-1), as described above, was tested for transfection efficiency in the following different cell lines: human breast cancer cells (MDA-231), rat bone marrow stromal cells (BMSC), human lung cancer cells (A549), African green monkey kidney epithelial cells (Vero), human ovarian cancer cells (HeLa) and human hepatocytes (HepG2). FIG. 16 shows the results of the cell lines transfected with gWIZ-GFP plasmid alone and transfected with gWIZ-GFP plasmid complexed with either sample 1015-1 (DETA-Dextran) or the commercially available transfection reagent, Escort™ IV (Sigma). The plasmid concentration was 1.3 μg/mL in the transfection medium.

As shown in FIG. 16, DETA-dextran (1015-1) showed transfection in all cell lines. Further, in all cell lines tested, dextran showed a higher transfection efficiency relative to the Escort™ IV (Sigma) transfection reagent.

### Example 12

### Using Different Concentrations of Aminated Dextran

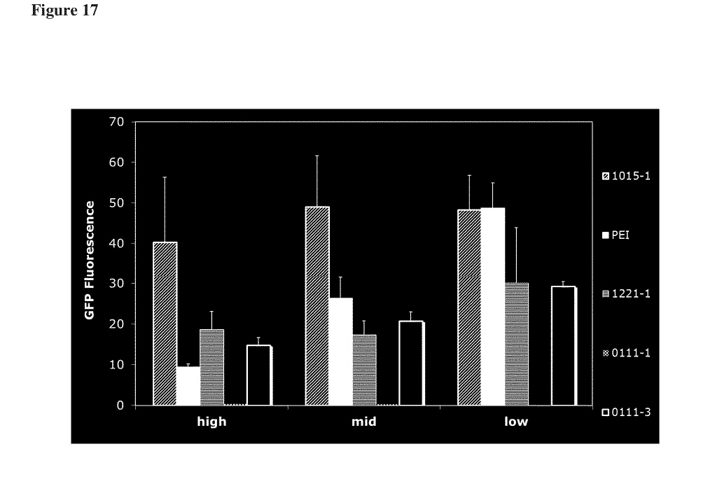

Dextran aminated with DETA (sample 1015-1), as described above, was complexed with gWIZ-GFP plasmid and expressed in 293T cells at three different concentrations: 16 μg/mL (high concentration), 8 μg/mL (middle concentration) and 4 μg/mL (low concentration). Transfection efficiency of aminated dextran at the three different concentrations was compared to PEI at three different concentrations and samples 1221-1, 011101 and 0111-3 at three different concentrations (FIG. 17). The high, middle and low concentrations of PEI were 8, 4 and 2 μg/mL, respectively, and the high, middle and low concentrations of samples 1221-1, 0111-1 and 0111-3 were 16, 8 and 4 μg/mL, respectively.

At the high and middle concentration conditions, sample 1015-1 demonstrated the highest transfection efficiency amongst the different transfection reagents tested. At the low concentration condition, sample 1015-1 and PEI demonstrated similar transfection efficiencies, with PEI demonstrating only marginally greater transfection efficiency (not statistically significant). Samples 1221-1, 0111-1 and 0111-3 showed consistently lower transfection efficiencies at all three different concentrations as compared to sample 1015-1. PEI demonstrated considerably lower transfection efficiencies at the high and middle concentration conditions compared to sample 1015-1. These results demonstrate that sample 1015-1 retained efficient transfection at all three different concentrations.

### Example 13

### Overall Comparison of Transfection Reagents

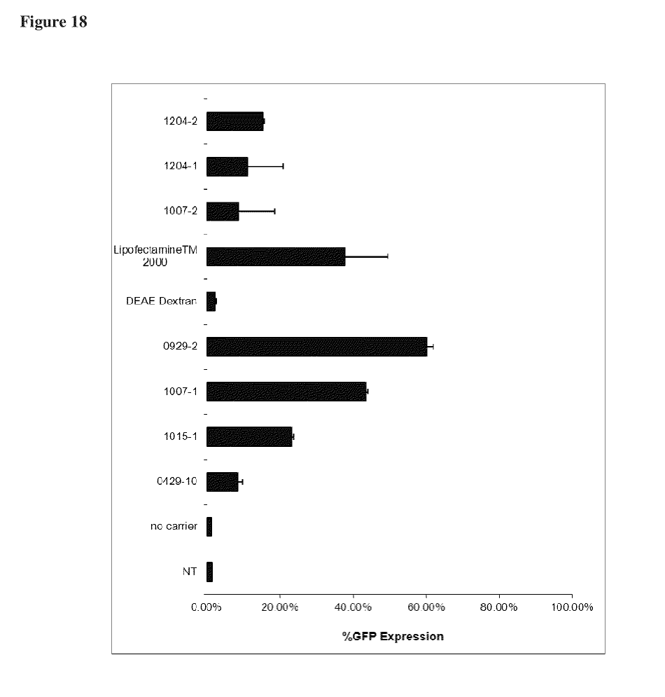

Selected aminated polymers of the present invention were expressed in 293T cells and compared to each other and other commercially available transfection reagents (Lipofectamine™ 2000 and DEAE-Dextran). All aminated polymer and transfection reagents were complexed with gWIZ-GFP plasmid and added to the cells at a concentration of 2 μg/mL (plasmid) and 10 μg/mL (carrier). The results are shown in FIG. 18.

All of the aminated polymers of the present invention demonstrated transfection ability in the 293T cells, as evidenced by significantly higher GFP fluorescence as compared to the no carrier control (i.e., gWIZ-GFP plasmid only) and the non-treated group (“NT”).

All citations are herein incorporated by reference, as if each individual publication was specifically and individually indicated to be incorporated by reference herein and as though it were fully set forth herein. Citation of references herein is not to be construed nor considered as an admission that such references are prior art to the present invention. Use of the term ‘a’ or ‘an’ includes both singular and plural references.

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A method of transfecting a cell with a nucleic acid, comprising contacting the cell with a composition comprising (1) a compound comprising a carbon polymer and one or more polyamine groups, wherein the carbon polymer is selected from the group consisting of hydroxyethylcellulose, dextran, poly(vinyl alcohol) and poly(methyl acrylate); and (2) a nucleic acid.

2. A method of introducing an exogenous nucleic acid into a cell, comprising contacting the cell with a composition comprising (1) a compound comprising a carbon polymer and one or more polyamine groups, wherein the carbon polymer is selected from the group consisting of hydroxyethylcellulose, dextran, poly(vinyl alcohol) and poly(methyl acrylate); and (2) a nucleic acid.

3. The method of claim 2 wherein the method is in vitro, ex vivo or in vivo.
